(19) United States
(12) Patent Application Publication
(10) Pub. No.: US 2025/0197473 A1
(43) Pub. Date: Jun. 19, 2025

(54) DUAL CHECKPOINT INHIBITORS AND METHODS OF USING THE SAME

(71) Applicant: SAGITTARIUS BIO, INC., Thousand Oaks, CA (US)

(72) Inventors: Kayvan Niazi, Thousand Oaks, CA (US); Sharooz Rabizadeh, Thousand Oaks, CA (US)

(21) Appl. No.: 18/842,271

(22) PCT Filed: Feb. 28, 2023
(86) PCT No.: PCT/US2023/014058

§ 371 Date: Aug. 28, 2024

Related U.S. Application Data
(60) Provisional application No. 63/314,861, filed on Feb. 28, 2022.

(51) Int. Cl. C07K 14/705; A61K 38/00; A61K 40/11; A61K 40/31; A61P 35/00; A61P 37/02; C12N 15/86

(52) U.S. Cl. CPC C07K 14/70596; A61K 40/11; A61K 40/31; A61P 35/00; A61P 37/02; C07K 14/70532; C12N 15/86; A61K 38/00; C07K 2319/30; C12N 2510/00; C12N 2710/10043

(57) ABSTRACT

The present application provides for polypeptides comprising immune checkpoint polypeptides, compositions comprising the same, and methods of using the same.

### CROSS-REFERENCE TO RELATED APPLICATIONS

This application claims priority to U.S. Provisional Application No. 63/314,861, filed on Feb. 28, 2022, which is hereby incorporated by reference in its entirety.

### SEQUENCE LISTING

This application contains a Sequence Listing filed electronically as an XML file, named “260034.000602 Sequence Listing XML,” which was created on Feb. 27, 2023, and is 50,550 bytes in size. The Sequence Listing is hereby incorporated by reference in its entirety.

### FIELD

Embodiments provided for herein relate to immune checkpoint inhibitors and methods of using the same.

### BACKGROUND

Immune checkpoint molecules are accessory molecules that either promote or inhibit T-cell activation. Two inhibitory molecules include cytotoxic T-lymphocyte antigen 4 (CTLA-4) and programmed cell death protein 1 (PD-1). PD-1 ligands (PD-L1 and PD-L2) expressed on cancer and other cells interact with PD-1 on T-cells to suppress T-cell killing of the tumor cells. Similarly, B7 family proteins (B7.1 and B7.2) expressed on cancer and other cells interact with CTLA-4 on T-cells and inhibit the activity of T-cells. Monoclonal antibody therapy has been developed against both PD-1 (Opdivo@) and CTLA-4 (Yervoy@) and have shown promise in pre-clinical and clinical studies for the treatment of tumors. However, the majority of patients do not develop sustained anti-tumor responses using these therapies. Thus, there is a need for improved therapies that take advantage of this critical immune checkpoint signaling nexus. The embodiments provided herein fulfill these needs as well as others.

### SUMMARY

In some embodiments, polypeptides are provided comprising from the N-terminus to the C-terminus a formula of X₁-L₁-X₂ or a formula of X₂-L₁-X₁. In some embodiments, X₁ is an immune checkpoint polypeptide or an active fragment thereof, such as but not limited to, a CTLA-4 polypeptide, a PD-1 polypeptide, a PD-L1 polypeptide, a PD-L2 polypeptide, a TIM3 polypeptide, a LAG3 polypeptide, a VISTA polypeptide, a SINGLEC7 polypeptide, a SINGLEC9 polypeptide, a TIGIT polypeptide, a CD96 polypeptide, a BTLA polypeptide, a B7H3 polypeptide, a B7H4 polypeptide, a CD155 polypeptide, a HHLA2 polypeptide, a BTN3A1 polypeptide, or an active fragment thereof. In some embodiments, X₂ is a B7.1 polypeptide or a B7.2 polypeptide. In some embodiments, L₁ is a polypeptide linker.

In some embodiments, polypeptides are provided comprising from the N-terminus to the C-terminus a formula of LS-X₁-L₁-X₂-L₂-X₃, LS-X₂-L₁-X₁-L₂-X₃, LS-X₁-L₁-X₃-L₂-X₂, or LS-X₂-L₁-X₃-L₂-X₁, wherein LS is a leader sequence; X₁ is a PD-L1 polypeptide or a PD-L2 polypeptide; X₂ is a B7.1 polypeptide or a B7.2 polypeptide; X₃ is a binder, such as a Fc domain, TCR, cytokine, cytokine trap, receptor ligand, or complement protein; L₁ is absent or is a polypeptide linker; and L₂ is absent or is a polypeptide linker.

In some embodiments, nucleic acid molecules are provided, which encode for a polypeptide as provided for herein.

In some embodiments, vectors are provided comprising a nucleic acid molecule as provided for herein. In some embodiments, the nucleic acid molecule encodes for a polypeptide as provided for herein.

In some embodiments, a plasmid or a composition is provided. In some embodiments, the plasmid or composition comprises a nucleic acid molecule as provided for herein. In some embodiments, the nucleic acid molecule encodes for a polypeptide as provided for herein.

In some embodiments, a pharmaceutical composition is provided. In some embodiments, the pharmaceutical composition comprises a polypeptide as provided for herein. In some embodiments, the pharmaceutical composition comprises a nucleic acid molecule as provided for herein. In some embodiments, the nucleic acid molecule encodes for a polypeptide as provided for herein.

In some embodiments, a recombinant virus is provided. In some embodiments, the recombinant virus comprises a nucleic acid molecule as provided for herein. In some embodiments, the nucleic acid molecule encodes for a polypeptide as provided for herein.

In some embodiments, a cell is provided. In some embodiments, the cell comprises a polypeptide as provided for herein. In some embodiments, the cell comprises a nucleic acid molecule as provided for herein. In some embodiments, the nucleic acid molecule encodes for a polypeptide as provided for herein.

In some embodiments, a method of producing a cell is provided. In some embodiments, the cell is a cell as provided for herein. In some embodiments, the method comprises contacting the cell with a vector comprising a nucleic acid molecule as provided for herein. In some embodiments, the nucleic acid molecule encodes for a polypeptide as provided for herein.

In some embodiments, a method of producing a polypeptide is provided. In some embodiments, the polypeptide is a polypeptide as provided for herein. In some embodiments, the method comprises transducing or transfecting a cell with a vector comprising a nucleic acid molecule as provided for herein. In some embodiments, the nucleic acid molecule encodes for a polypeptide as provided for herein.

In some embodiments, a method for modulating an immune response in a patient is provided. In some embodiments, the method comprises administering to the patient a pharmaceutical composition comprising a polypeptide as provided for herein. In some embodiments, the method comprises administering to the patient a pharmaceutical composition comprising a vector as provided for herein. In some embodiments, the vector comprises a nucleic acid molecule as provided for herein. In some embodiments, the nucleic acid molecule encodes for a polypeptide as provided for herein.

In some embodiments, a method for treating cancer in a subject is provided. In some embodiments, the method comprises administering to the patient a pharmaceutical composition comprising a polypeptide as provided for herein. In some embodiments, the method comprises administering to the patient a pharmaceutical composition comprising a vector as provided for herein. In some embodiments, the vector comprises a nucleic acid molecule as provided for herein. In some embodiments, the nucleic acid molecule encodes for a polypeptide as provided for herein.

In some embodiments, a method for treating a disease or disorder in a subject is provided. In some embodiments, the disease or disorder is a viral infection, a bacterial infection or a fungal infection, or a disease or disorder as provided for herein. In some embodiments, the method comprises administering to the patient a pharmaceutical composition comprising a polypeptide as provided for herein. In some embodiments, the method comprises administering to the patient a pharmaceutical composition comprising a vector as provided for herein. In some embodiments, the vector comprises a nucleic acid molecule as provided for herein. In some embodiments, the nucleic acid molecule encodes for a polypeptide as provided for herein.

### DETAILED DESCRIPTION

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or 10%, ±5%, 1%, or ±0.1% from the specified value, as such variations are appropriate to perform the disclosed methods.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists of.”

As used herein, the term “contacting” means bringing together of two elements in an in vitro system or an in vivo system. For example, “contacting” a vector with a cell or with an individual or patient or cell includes the administration of the vector to an individual or patient, such as a human, as well as, for example, introducing a compound into a sample containing a cellular or purified preparation containing the cell. As used herein, “contacting” can be synonymous with “delivering.” As used herein, “contacting” or “delivering” encompasses all necessary steps or methods. For example, “contacting” a vector with a cell would comprise nucleoporation, transfection, viral delivery, and the like.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, an amount that when administered to a mammal, causes a detectable level of immune cell activation compared to the immune cell activation detected in the absence of the composition. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another, or another domain or peptide sequence can be between the two domains or sequences and such sequences would still be considered to be fused or linked to one another. In some embodiments, the various domains or proteins provided for herein are linked or fused directly to one another or a linker sequences, such as a glycine/serine sequence link the two domains together.

“Heterologous” as used herein refers to a non-native nucleic acid or amino acid sequence that is introduced into a cell, organism, or system. The nucleic acid sequence can comprise a polynucleotide of any length. The amino acid sequence can comprise a peptide or polypeptide of any length.

“Identity” as used herein refers to the subunit sequence identity between two polymeric molecules such as between two nucleic acid or amino acid molecules, such as, between two polynucleotide or polypeptide molecules. When two amino acid sequences have the same residues at the same positions, e.g., if a position in each of two polypeptide molecules is occupied by an Arginine, then they are identical at that position. The identity or extent to which two amino acid or two nucleic acid sequences have the same residues at the same positions in an alignment is often expressed as a percentage. The identity between two amino acid or two nucleic acid sequences is a direct function of the number of matching or identical positions; e.g., if half of the positions in two sequences are identical, the two sequences are 50% identical; if 90% of the positions (e.g., 9 of 10), are matched or identical, the two amino acids sequences are 90% identical.

As used herein “located” is meant to give positional clarity in an amino acid or nucleic acid sequence. For example, a sequence X that is said to be located between a first portion A and a second portion B would yield the potential formulas A-X-B or B-X-A.

In a similar manner, the term “upstream” is meant to give further positional clarity in a nucleic acid or polynucleotide sequence. In the context of a polynucleotide sequence, a sequence X that is said to be located upstream of a first portion A would indicate that the sequence X is located prior to portion A such that the formula would read 5′-X-A-3′.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups; glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e3 and el00 indicating a closely related sequence. In some embodiments, sequence identity is determined by using BLAST with the default settings.

To the extent embodiments provided for herein, includes composition comprising various proteins, these proteins may, in some instances, comprise amino acid sequences that have sequence identity to the amino acid sequences disclosed herein. Therefore, in certain embodiments, depending on the particular sequence, the degree of sequence identity is preferably greater than 50% (e.g., 60%, 70%, 75%, 80%, 85%, 90%, 91%, 92%, 93%, 94%, 95%, 96%, 97%, 98%, 99% or more) to the SEQ ID NOs disclosed herein. These proteins may include homologs, orthologues, allelic variants, and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty—12 and gap extension penalty=1.

These proteins may, compared to the disclosed proteins, include one or more (e.g., 1, 2, 3,4, 5, 6, 7, 8, 9, 10, etc.) conservative amino acid replacements i.e., replacements of one amino acid with another which has a related side chain. Genetically-encoded amino acids are generally divided into four families: (1) acidic i.e., aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The proteins may have one or more (e.g., 1, 2, 3, 4, 5, 6, 7, 8, 9, 10, etc.) single amino acid deletions relative to the disclosed protein sequences. The proteins may also include one or more (e.g., 1, 2, 3, 4, 5, 6, 7, 8, 9, 10, etc.) insertions (e.g., each of 1, 2, 3, 4 or 5 amino acids) relative to the disclosed protein sequences.

As used herein, the phrase “in vivo” in reference to a cell being transduced, transfected, or transformed in vivo, refers to a cell being transduced, transfected, or transformed in the subject without the cells being removed from the subject before such cells are transduced, transfected or transformed.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell as provided herein. Molecules may be modified in many ways, including chemically, structurally, and functionally, such as mutations, substitutions, insertions, or deletions (e.g., internal deletions truncations). Cells may be modified through the introduction of nucleic acids or the expression of heterologous proteins.

By the term “modulating,” as used herein, is meant mediating an increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

As used herein, the following abbreviations for the commonly occurring nucleic acid bases are used: “A” refers to adenosine, “C” refers to cytosine, “G” refers to guanosine, “T′” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

The term “oligonucleotide” typically refers to short polynucleotides. It will be understood that when a nucleotide sequence is represented by a DNA sequence (i.e., A, T, C, G), this also provides the corresponding RNA sequence (i.e., A, U, C, G) in which “U” replaces “T.”

The term “polynucleotide” as used herein is defined as a chain of nucleotides. Furthermore, nucleic acids are polymers of nucleotides. Thus, the terms “nucleic acids” and “polynucleotides” as used herein are interchangeable. As used herein polynucleotides include, but are not limited to, all nucleic acid sequences which are obtained by any methods available in the art, including, without limitation, recombinant methods, i.e., the cloning of nucleic acid sequences from a recombinant library or a cell genome, using cloning technology and PCR, and the like, and by synthetic means.

As used herein, the terms “peptide,” “polypeptide,” and “protein” are used interchangeably, and refer to a compound comprised of a plurality of amino acid residues covalently linked by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “subject” includes living organisms, including those in which an immune response can be elicited (e.g., mammals). As used herein, the term “subject” or “patient” or “individual” may be used interchangeably. A “subject,” as used herein, may be a human or non-human mammal. Non-human mammals include, for example, livestock and pets, such as ovine, bovine, porcine, canine, non-human primates, feline and murine mammals. In some embodiments, the subject is human.

As used herein, the phrase “in need thereof” means that the subject (animal or mammal) has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into a cell. A “transfected” or “transformed” or “transduced” cell is one which has been transfected, transformed, or transduced with exogenous nucleic acid. The cell includes the primary subject cell and its progeny. In some embodiments, the transfection, transformation, or transduction is performed or occurs in vivo.

To “treat” a disease as the term is used herein, means to reduce the frequency or severity of at least one sign or symptom of a disease or disorder experienced by a subject.

As used herein, the term “variant” when used in conjunction to an amino acid sequence refers to a sequence that is at least, or about, 85%, 90%, 91%, 92%, 93%, 94%, 95%, 96%, 97%, 98%, or 99% identical to the reference sequence. In some embodiments, the variant comprises 1, 2, 3, 4, 5, 6, 7, 8, 9, or 10 substitutions. In some embodiments, the substitution is a conservative substitution.

A “vector” is a composition of matter which comprises an isolated nucleic acid encoding a protein or a peptide. Numerous vectors are known in the art including, but not limited to, linear polynucleotides, plasmids, DNA, and RNA. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the embodiments can be presented in a range format. The description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

Without being bound to any particular theory, the embodiments provided for herein have been found to simultaneously inhibit multiple immune checkpoint proteins with the use of a single polypeptide molecule. Current immune checkpoint treatment strategies require the administration of multiple immune checkpoint targeting molecules to target more than one immune checkpoint protein. While the use of combination treatment using these single immune checkpoint targeting protein molecules has demonstrated increased clinical benefit compared to mono-therapy treatments, the risk of severe or dose-limiting adverse or long term systemic off-target immunological events are also increased. By utilizing a single molecule, the adverse and off target events may be controlled. Further, incorporation of binding domains into the polypeptide of embodiments provided for herein will provide targeted delivery of the immune checkpoint inhibitor polypeptide, further increasing efficacy and decreasing undesired events.

### Compositions

Accordingly, described herein are polypeptide molecules comprising one or more immune checkpoint inhibitors. In some embodiments, a polypeptide is provided, the polypeptide comprising from the N-terminus to the C-terminus a formula of X₁-L₁-X₂ or a formula of X₂-L₁-X₁, wherein X₁ and X₂ are each, independently, immune checkpoint inhibiting polypeptides, and L₁ is a polypeptide linker. In some embodiments, X₁ and X₂ comprise the same immune checkpoint inhibiting polypeptide. In some embodiments, X₁ and X₂ comprise different immune checkpoint inhibiting polypeptides. In some embodiments, X₁ and X₂ can be any polypeptide or fragment thereof that would inhibit an immune checkpoint protein. In some embodiments, X₁ and X₂ target the same immune checkpoint protein. In some embodiments, X₁ and X₂ target different immune checkpoint proteins. In some embodiments, X₁ and X₂ target immune checkpoint proteins are selected from the group including, but not limited to, CTLA-4, PD-1, PD-L1, TIM3, LAG3, VISTA, SIGLEC7, SIGLEC9, TIGIT, CD96, BTLA, B7H3, B7H4, CD155, HHLA2, and BTN3A1.

In some embodiments, the polypeptide comprises from the N-terminus to the C-terminus a formula of X₁-L₁-X₂. In some embodiments, the polypeptide comprises from the N-terminus to the C-terminus a formula of X₂-L₁-X₁. In some embodiments, the polypeptide comprises from the N-terminus to the C-terminus a formula of X₁-L₁-X₁. In some embodiments, the polypeptide comprises from the N-terminus to the C-terminus a formula of X₂-L₁-X₂.

In some embodiments, X₁ is a peptide selected from the group comprising a CTLA-4 polypeptide, a PD-1 polypeptide, PD-L1 polypeptide, a PD-L2 polypeptide, a B7.1 polypeptide, or a B7.2 polypeptide, a TIM3 polypeptide, a LAG3 polypeptide, a VISTA polypeptide, a SINGLEC7 polypeptide, a SINGLEC9 polypeptide, a TIGIT polypeptide, a CD96 polypeptide, a BTLA polypeptide, a B7H3 polypeptide, a B7H4 polypeptide, a CD155 polypeptide, a HHLA2 polypeptide, a BTN3A1 polypeptide, or an active fragment thereof. In some embodiments, X₂ is a peptide selected from the group comprising a CTLA-4 polypeptide, a PD-1 polypeptide, PD-L1 polypeptide, a PD-L2 polypeptide, a B7.1 polypeptide, or a B7.2 polypeptide, a TIM3 polypeptide, a LAG3 polypeptide, a VISTA polypeptide, a SINGLEC7 polypeptide, a SINGLEC9 polypeptide, a TIG1T polypeptide, a CD96 polypeptide, a BTLA polypeptide, a B7H3 polypeptide, a B7H4 polypeptide, a CD155 polypeptide, a HHLA2 polypeptide, a BTN3A1 polypeptide, or an active fragment thereof. In some embodiments, both X₁ and X₂ are each, independently, peptides selected from the group comprising a PD-L1 polypeptide, a PD-L2 polypeptide, a B7.1 polypeptide, or a B7.2 polypeptide. In some embodiments X₁ and X₂ comprise the same polypeptide. In some embodiments, X₁ and X₂ comprise different polypeptides.

In some embodiments, X₁ is a PD-L1 polypeptide, and X₂ is a B7.1 polypeptide. In some embodiments, X₁ is a PD-L1 polypeptide, and X₂ is a B7.2 polypeptide. In some embodiments, X₁ is a PD-L2 polypeptide, and X₂ is a B7.1 polypeptide. In some embodiments, X₁ is a PD-L2 polypeptide, and X₂ is a B7.2 polypeptide.

In some embodiments, the PD-L1 polypeptide comprises an amino acid sequence of SEQ ID NO. 1: MRIFAVFIFMTYWHLLNAFTVTVPKDLYVVEYGSNMTIECKFPVEKQLDLAALIVYWE MEDKNIIQFVHGEEDLKVQHSSYRQRARLLKDQLSLGNAALQITDVKLQDAGVYRCMI SYGGADYKRITVKVNAPYNKINQRILVVDPVTSEHELTCQAEGYPKAEVIWTSSDHQVL SGKTTITNSKREEKLFNVTSTLRINTTTNEIFYCTFRRLDPEENHTAELVIPELPLAHPPNE RTHLVILGAILLCLGVALTFIFRLRKGRMMDVKKCGIQDTNSKKQSDTHLEET (SEQ ID NO. 1) or an active fragment thereof. In any of the embodiments herein, the PD-L1 polypeptide may comprise an amino acid sequence corresponding to the entire PD-L1 protein, or to any fragment thereof. Further, it is to be understood that the PD-L1 polypeptide is not limited to a specific isoform, but rather the PD-L1 polypeptide as recited herein encompasses all PD-L1 isoforms. The PD-L1 polypeptide as presented herein can comprise the wild-type PD-L1 protein, or it can comprise any PD-L1 mutant protein known in the art. Further, it is to be understood that the PD-L1 polypeptide is not limited to the human PD-L1 polypeptide, but rather encompasses PD-L1 polypeptide sequences from other relevant species, including but not limited to mouse, rat, non-human primate, bovine, equine, and the like.

In some embodiments, the PD-L1 polypeptide comprises a PD-L1 extracellular domain. In some embodiments, the PD-L1 polypeptide comprises an amino acid sequence substantially similar to SEQ ID NO. 2. FTVTVPKDLYVVEYGSNMTIECKFPVEKQLDLAALIVYWEMEDKNIIQFVHGEEDLKVQ HSSYRQRARLLKDQLSLGNAALQITDVKLQDAGVYRCMISYGGADYKRITVKVNAPYN KINQRILVVDPVTSEHELTCQAEGYPKAEVIWTSSDHQVLSGKTITTNSKREEKLFNVTS TLRINTTTNEIFYCTFRRLDPEENHTAELVIPELPLAHPPNER (SEQ ID NO. 2). In some embodiments, the PD-L1 polypeptide comprises an active fragment of SEQ ID NO. 2. In some embodiments, the PD-L1 polypeptide comprises an amino acid sequence having at least 60% identity to SEQ ID NO. 2. In some embodiments, the PD-L1 polypeptide comprises an amino acid sequence having at least 65%, 70%, 75%, 80%, 85%, 86%, 87%, 88%, 89%, 909%, 91%, 92%, 93%, 94%, 95%, 96%, 97%, 98%, or 99% identity to SEQ ID NO. 2. In some embodiments, the PD-L1 polypeptide comprises an amino acid sequence identical to SEQ ID NO. 2.

In some embodiments, the PD-L2 polypeptide comprises an amino acid sequence of SEQ ID NO. 3: MIFLLLMLSLELQLHQTAALFTVTVPKELYIIEHGSNVTLECNFDTGSHVNLGAITASLQK VENDTSPHRERA TLLEEQLPLGKASFHIPQVQVRDEGQYQCIIIYGVAWDYKYLTLKVK A SYRKINTHILKVPETDEVELTCQATGYPLAEVSWPNVSVPANTSHSRTPEGLYQVTSVL RLKPPPGRNFSCVFWNTHVRELTLASIDLQSQMEPRTHPTWLLHIFIPFCIIAFIFIATVIAL RKQLCQKLYSSKDTTKRPVTTTKREVNSAI (SEQ ID NO. 3) or an active fragment thereof.

In any of the embodiments herein, the PD-L2 polypeptide may comprise an amino acid sequence corresponding to the entire PD-L2 protein, or to any fragment thereof. Further, it is to be understood that the PD-L2 polypeptide is not limited to a specific isoform, but rather the PD-L2 polypeptide as recited herein encompasses all PD-L2 isoforms. The PD-L2 polypeptide as presented herein can comprise the wild-type PD-L2 protein, or it can comprise any PD-L2 mutant protein known in the art. Further, it is to be understood that the PD-L2 polypeptide is not limited to the human PD-L1 polypeptide, but rather encompasses PD-L2 polypeptide sequences from other relevant species, including but not limited to mouse, rat, non-human primate, bovine, equine, and the like.

In some embodiments, the PD-L2 polypeptide comprises a PD-L2 extracellular domain. In some embodiments, the PD-L2 polypeptide comprises an amino acid sequence substantially similar to SEQ ID NO. 4. LFTVTVPKELYIIEHGSNVTLECNFDTGSHVNLGAITASLQKVENDTSPHRERATLLEEQL PLGKASFHIPQVQVRDEGQYQCUIYGVAWDYKYLTLKVKASYRKINTHILKVPETDEVE LTCQATGYPLAEVSWPNVSVPANTSHSRTPEGLYQVTSVLRLKPPPGRNFSCVFWNTHV RELTLASIDLQSQMEPRTHPT (SEQ ID NO. 4). In some embodiments, the PD-L2 polypeptide comprises an active fragment of SEQ ID NO. 4. In some embodiments, the PD-L2 polypeptide comprises an amino acid sequence having at least 60% identity to SEQ ID NO. 4. In some embodiments, the PD-L2 polypeptide comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 4. In some embodiments, the PD-L2 polypeptide comprises an amino acid sequence identical to SEQ ID NO. 4.

In some embodiments, the B7.1 polypeptide comprises an amino acid sequence of SEQ ID NO. 5: MGHTRRQGTSPSKCPYLNFFQLLVLAGLSHFCSGVIHVTKEVKEVATLSCGHNVSVEEL AQTRIYWQKEKKMVLTMMSGDMNIWPEYKNRTIFDITNNLSIVILALRPSDEGTYECVV LKYEKDAFKREHLAEVTLSVKADFPTPSISDFEIPTSNIRRUHCSTSGGFPEPHLSWLENGE ELNAINTTVSQDPETELYAVSSKLDFNMTTNHSFMCLIKYGHLRVNQTFNWNTTKQEHF PDNLLPSWAITLISVNGIFVICCLTYCFAPRCRERRRNERLRRESVRPV (SEQ ID NO. 5) or an active fragment thereof. In any of the embodiments herein, the B7.1 polypeptide may comprise an amino acid sequence corresponding to the entire B7.1 protein, or to any fragment thereof. Further, it is to be understood that the B7.1 polypeptide is not limited to a specific isoform, but rather the B7.1 polypeptide as recited herein encompasses all B7.1 isoforms. The B7.1 polypeptide as presented herein can comprise the wild type B7.1 protein, or it can comprise any B7.1 mutant protein known in the art. Further, it is to be understood that the B7.1 polypeptide is not limited to the human B7.1 polypeptide, but rather encompasses B7.1 polypeptide sequences from other relevant species, including but not limited to mouse, rat, non-human primate, bovine, equine, and the like.

In some embodiments, the B7.1 polypeptide comprises a B7.1 extracellular domain. In some embodiments, the B7.1 polypeptide comprises an amino acid sequence substantially similar to SEQ ID NO. 6. VIHVTKEVKEVATLSCGHNVSVEELAQTRIYWQKEKKMVLTMMSGDMNIWPEYKNRT IFDITNNLSIVILALRPSDEGTYECVVLKYEKDAFKREHLAEVTLSVKADFPTPSISDFEIPT SNIRRIICSTSGGFPEPHLSWLENGEELNAINTTVSQDPETELYAVSSKLDFNMTTNHSFM CLIKYGHLRVNQTFNWNTTKQEHFPDN (SEQ ID NO. 6). In some embodiments, the B7.1 polypeptide comprises an active fragment of SEQ ID NO. 6. In some embodiments, the B7.1 polypeptide comprises an amino acid sequence having at least 60% identity to SEQ ID NO. 6. In some embodiments, the B7.1 polypeptide comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 6. In some embodiments, the B7.1 polypeptide comprises an amino acid sequence identical to SEQ ID NO. 6.

In some embodiments, the B7.2 polypeptide comprises an amino acid sequence of SEQ ID NO. 7: MDPQCTMGLSNILFVMAFLLSGAAPLKIQAYFNETADLPCQFANSQNQSLSELVVFWQD QENLVLNEVYLGKEKFDSVHSKYMGRTSFDSDSWTLRLHNLQIKDKGLYQCHHHKKPT GMIRIHQMNSELSVLANFSQPEIVPISNITENVYINLTCSSIHGYPEPKKMSVLLRTKNSTI EYDGVMQKSQDNVTELYDVSISLSVSFPDVTSNMTIFCILETDKTRLLSSPFSIELEDPQPP PDHIPWITAVLPTVITCVMVFCLILWKWKKKKRPRNSYKCGTNTMEREESEQTKKREKIH IPERSDEAQRVFKSSKTSSCDKSDTCF (SEQ ID NO. 7) or an active fragment thereof. In any of the embodiments herein, the B7.2 polypeptide may comprise an amino acid sequence corresponding to the entire B7.2 protein, or to any fragment thereof. Further, it is to be understood that the B7.2 polypeptide is not limited to a specific isoform, but rather the B7.2 polypeptide as recited herein encompasses all B7.2 isoforms. The B7.2 polypeptide as presented herein can comprise the wild type B7.2 protein, or it can comprise any B7.2 mutant protein known in the art. Further, it is to be understood that the B7.2 polypeptide is not limited to the human B7.2 polypeptide, but rather encompasses B7.2 polypeptide sequences from other relevant species, including but not limited to mouse, rat, non-human primate, bovine, equine, and the like.

In some embodiments, the B7.2 polypeptide comprises a B7.2 extracellular domain. In some embodiments, the B7.2 polypeptide comprises an amino acid sequence substantially similar to SEQ ID NO. 8. APLKIQAYFNETADLPCQFANSQNQSLSELVVFWQDQENLVLNEVYLGKEKFDSVHSK YMGRTSFDSDSWTLRLHNLQIKDKGLYQCIIHHKKPTGMIRIHQMNSELSVLANFSQPEI VPISNITENVYINLTCSSIHGYPEPKKMSVLLRTKNSTIEYDGVMQKSQDNVTELYDVSIS LSVSFPDVTSNMTIFCILETDKTRLLSSPFSIELEDPQPPPDHIP (SEQ ID NO. 8). In some embodiments, the B7.2 polypeptide comprises an amino acid sequence having at least 60% identity to SEQ ID NO. 8. In some embodiments, the B7.2 polypeptide comprises an amino acid sequence having at least 65%, 70%, 75%, 80%, 85%, 86%, 87%, 88%, 89%, 90%, 91%, 92%, 93%, 940%, 95%, 96%, 97%, 98%, or 99% identity to SEQ ID NO. 8. In some embodiments, the B7.2 polypeptide comprises an amino acid sequence identical to SEQ ID NO. 8.

The linker, L₁, can comprise any peptide linker. In some embodiments, the peptide linker comprises an amino acid sequence of GSGSGGGSGSGGGSGSGSG (SEQ ID NO 9). In some embodiments, the peptide linker comprises an amino acid sequence of substantially similar to SEQ ID NO 9. In some embodiment, the peptide linker comprises a fragment of SEQ ID NO 9, or comprises n repeats of SEQ ID NO 9, wherein n is an integer from 1-5. In some embodiments, the linker is a flexible linker. In some embodiments, the linker is a rigid linker. In some embodiments, the linker can be as described herein or as illustrated in Table 1 below.

TABLE 1

| Type | Sequence | SEQ ID NO: |
|---|---|---|
| flexible | GGGGS | 23 |
| flexible | GGGGGGGGS | 24 |
| flexible | GGGGSGGGGSGGGGS | 25 |
| flexible | GGGGSGGGGSGGGGSGGGGS | 26 |
| flexible | GGGGGG | 27 |
| flexible | GGGGGGGG | 28 |
| rigid | EAAAKEAAAKEAAAK | 29 |
| rigid | EAAK | 30 |
| rigid | EAAKEAAK | 31 |
| rigid | EAAKEAAKEAAK | 32 |
| rigid | A(EAAAK)₄ALEA(EAAAK)₄A | 33 |
| rigid | AEAAAKEAAAKA | 34 |
| rigid | PAPAP | 35 |
| rigid | (Ala-Pro)ₙ (10-34 aa) | — |
| cleavable | disulfide | — |
| cleavable | VSQTSKLTRAETVFPDV | 36 |
| cleavable | PLGLWA | 37 |
| cleavable | RVLAEA | 38 |
| cleavable | EDVVCCSMSY | 39 |
| cleavable | GGIEGRGS | 40 |
| cleavable | TRHRQPRGWE | 41 |
| cleavable | AGNRVRRSVG | 42 |
| cleavable | RRRRRRRRR | 43 |
| cleavable | GFLG | 44 |
| dipeptide | LE | — |

In some embodiments, the polypeptide provided further comprises a leader sequence. In some embodiments, the leader sequence is located at the N-terminus of the polypeptide. In some embodiments, the leader sequence is a targeting polypeptide sequence. In some embodiments, the targeting sequence is used to direct the polypeptide to a specific subcellular compartment or organelle. In some embodiments, the targeting sequence is used to direct the polypeptide to the nucleus. In some embodiments, the leader sequence is a cell membrane targeting sequence. In some embodiments, the leader sequence is native to the polypeptide comprising the N-terminus of the polypeptide. In some embodiments, the leader sequence is not native to the polypeptide comprising the N-terminus of the polypeptide. In some embodiments, the leader sequence comprises a polypeptide sequence corresponding to the cell membrane targeting domain of PD-L1. In some embodiments, the leader sequence comprises a polypeptide sequence corresponding to the cell membrane targeting domain of PD-L2. In some embodiments, the leader sequence comprises a polypeptide sequence corresponding to the cell membrane targeting domain of B7.1. In some embodiments, the leader sequence comprises a polypeptide sequence corresponding to the cell membrane targeting domain of B7.2. In some embodiments, the leader sequence comprises a polypeptide sequence corresponding to any cell membrane targeting domain that will deliver the provided polypeptide to the cell surface.

In some embodiments, the leader sequence comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO. 10 or is identical to SEQ ID NO. 10:

(SEQ ID NO. 10)
MIFLLLMLSLELQLHQIAA

or is an active fragment thereof.

In some embodiments, the leader sequence comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO. 11 or is identical to SEQ ID NO. 11:

(SEQ ID NO. 11)
MRIFAVFIFMTYWHLLNA

or is an active fragment thereof.

In some embodiments, the leader sequence comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO. 12 or is identical to SEQ ID NO. 12:

(SEQ ID NO. 12)
MGHTRRQGTSPSKCPYLNFFQLLVLAG

or is an active fragment thereof.

In some embodiments, the leader sequence comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO. 13 or is identical to SE ID NO. 13:

(SEQ ID NO. 13)
MDPQCTMGLSNILFVMAFLLSGA

or is an active fragment thereof.

In some embodiments, the provided polypeptide further comprises a targeting domain. The targeting domain can comprise any protein that will direct the provided polypeptide to its intended cellular target. In some embodiments, the targeting domain directs the provided polypeptide to an antigen presenting cell, an immune cell, a cancer cell, an epithelial cell, a mesenchymal cell, a neuron, and the like. In some embodiments, the targeting domain comprises a protein from the group including, but not limited to, a cytokine, a cytokine trap, a receptor ligand, a complement protein, a T-cell receptor, an antibody, an Fc domain, a chimeric antigen receptor, an antigen, a tumor antigen, and the like, or any fragment thereof.

In some embodiments, a polypeptide is provided, the polypeptide comprising from the N-terminus to the C-terminus a formula of selected from the group comprising LS-X₁-L₁-X₂-L₂-X₃, LS-X₁-L₁-X₃-L₂-X₂, LS-X₂-L₁-X₁-L₂-X₃, or LS-X₂-L₁-X₃-L₂-X₁, wherein LS is a leader sequence, X₁ and X₂ are each, independently, immune checkpoint inhibiting polypeptides, X₃ is a targeting domain, and L₁ and L₂ are each, independently, polypeptide linkers. In some embodiments, X₁ and X₂ comprise the same immune checkpoint inhibiting polypeptide. In some embodiments, X₁ and X₂ comprise different immune checkpoint inhibiting polypeptides. In some embodiments, X₁ and X₂ can be any polypeptide or fragment thereof that would inhibit an immune checkpoint protein. In some embodiments, X₁ and X₂ target the same immune checkpoint protein. In some embodiments, X₁ and X₂ target different immune checkpoint proteins. In some embodiments, X₁ and X₂ target immune checkpoint proteins selected from the group including, but not limited to, CTLA-4, PD-1, PD-L1, TIM3, LAG3, VISTA, SIGLEC7, SIGLEC9, TIGIT, CD96, BTLA, B7H3, B7H4, CD155, HHLA2, and BTN3A1. In some embodiments, L₁ and L₂ comprise the same polypeptide linker. In some embodiments, L₁ and L₂ comprise different polypeptide linkers. In some embodiments, L₁ and L₂ are each, independently a polypeptide linker as provided for herein. In some embodiments, the targeting domain X₃ can comprise any protein that will direct the provided polypeptide to its intended cellular target. In some embodiments, the targeting domain directs the provided polypeptide to an antigen presenting cell, an immune cell, a cancer cell, an epithelial cell, a mesenchymal cell, a neuron, and the like. In some embodiments, the targeting domain comprises a protein from the group including, but not limited to, a cytokine, a cytokine trap, a receptor ligand, a complement protein, a T-cell receptor, an antibody, an Fc domain, a chimeric antigen receptor, an antigen, a tumor antigen, and the like, or any fragment thereof.

In some embodiments, the polypeptide comprises from the N-terminus to the C-terminus a formula of LS-X₁-L₁-X₂-L₂-X₃. In some embodiments, the polypeptide comprises from the N-terminus to the C-terminus a formula of LS-X₁-L₁-X₃-L₂-X₂. In some embodiments, the polypeptide comprises from the N-terminus to the C-terminus a formula of LS-X₂-L₁-X₁-L₂-X₃.

In some embodiments, the polypeptide comprises from the N-terminus to the C-terminus a formula of LS-X₂-L₁-X₃-L₂-X₁. In some embodiments, LS is a leader sequence as provided for herein. In some embodiments, X₁ comprises a PD-L1 polypeptide or a PD-L2 polypeptide as provided for herein. In some embodiments, X₂ comprises a B7.1 polypeptide or a B7.2 polypeptide as provided for herein. In some embodiments, the targeting domain X₃ is a targeting domain as provided for herein. In some embodiments, L₁ and L₂ are each, independently, absent or a polypeptide linker as provided for herein.

In some embodiments, X₁ is a PD-L1 polypeptide as provided for herein; X₂ is a B7.1 polypeptide as provided for herein; X₃ is an antibody Fc domain; L₁ is a polypeptide linker as provided for herein; and L₂ is a polypeptide linker as provided for herein or absent.

In some embodiments, X₁ is a PD-L1 polypeptide as provided for herein; X₂ is a B7.2 polypeptide as provided for herein; X₃ is an antibody Fc domain; L₁ is a polypeptide linker as provided for herein; and L₂ is a polypeptide linker as provided for herein or absent.

In some embodiments, X₁ is a PD-L2 polypeptide as provided for herein; X₂ is a B7.1 polypeptide as provided for herein; X₃ is an antibody Fc domain; L₁ is a polypeptide linker as provided for herein; and L₂ is a polypeptide linker as provided for herein or absent.

In some embodiments, X₁ is a PD-L2 polypeptide as provided for herein; X₂ is a B7.2 polypeptide as provided for herein; X₃ is an antibody Fc domain; L₁ is a polypeptide linker as provided for herein; and L₂ is a polypeptide linker as provided for herein or absent.

In some embodiments, the PD-L1 polypeptide comprises a PD-L1 extracellular domain as provided for herein. In some embodiments, the PD-L1 polypeptide comprises an amino acid sequence substantially similar to SEQ ID NO. 2. In some embodiments, the PD-L1 polypeptide comprises an amino acid sequence having at least 60% identity to SEQ ID NO. 2. In some embodiments, the PD-L1 polypeptide comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 2. In some embodiments, the PD-L1 polypeptide comprises an amino acid sequence identical to SEQ ID NO. 2.

In some embodiments, the PD-L2 polypeptide comprises a PD-L2 extracellular domain as provided for herein. In some embodiments, the PD-L2 polypeptide comprises an amino acid sequence substantially similar to SEQ ID NO. 4. In some embodiments, the PD-L2 polypeptide comprises an amino acid sequence having at least 60% identity to SEQ ID NO. 4. In some embodiments, the PD-L2 polypeptide comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 4. In some embodiments, the PD-L2 polypeptide comprises an amino acid sequence identical to SEQ ID NO. 4.

In some embodiments, the B7.1 polypeptide comprises a B7.1 extracellular domain as provided for herein. In some embodiments, the B7.1 polypeptide comprises an amino acid sequence substantially similar to SEQ ID NO. 6. In some embodiments, the B7.1 polypeptide comprises an amino acid sequence having at least 60% identity to SEQ ID NO. 6. In some embodiments, the B7.1 polypeptide comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 6. In some embodiments, the B7.1 polypeptide comprises an amino acid sequence identical to SEQ ID NO. 6.

In some embodiments, the B7.2 polypeptide comprises a B7.2 extracellular domain as provided for herein. In some embodiments, the B7.2 polypeptide comprises an amino acid sequence substantially similar to SEQ ID NO. 8. In some embodiments, the B7.2 polypeptide comprises an amino acid sequence having at least 60% identity to SEQ ID NO. 8. In some embodiments, the B7.2 polypeptide comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 8. Tn some embodiments, the B7.2 polypeptide comprises an amino acid sequence identical to SEQ ID NO. 8.

In some embodiments, the homodimerization domain comprises any immunoglobulin fold containing protein. In some embodiments, the immunoglobulin fold containing protein comprises any human Fc domain. In some embodiments, the Fc domain is selected from the group including, but not limited to IgG, IgM, IgA, IgE, or IgD, or any subclass thereof, including but not limited to IgG₁, IgG₂, IgG₃, IgG₄, IgA₁ or IgA₂. In some embodiments, the Fc domain can be engineered to enhance binding to a specific target protein. For example, in some embodiments, the Fc domain is bispecific. In some embodiments, the Fc domain is engineered to enhance binding to a specific Fc receptor (FcR). In some embodiments, the FcR is selected from the group including, but not limited to FcγR, FcαR, FcεR, FcγRI, FcγRIIA, FcγRIIB1, FcγRIIB2, FcγRIIIA, FcγRIIIB, FcεRI, FcεRII, FcαRI, FcαR/μR, or FcRn. Specific engineering/point mutations of an Fc domain to enhance binding to a specific FcR are known in the art and are within the scope of this application.

In some embodiments, the Fc domain is a human IgG1 Fc domain. In some embodiments, the Fc domain comprises an amino acid sequence substantially similar to SEQ ID NO. 14: DKTHTCPPCPAPELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO. 14) or an active fragment thereof. In some embodiments, the Fc domain comprises an amino acid sequence having at least 60% identity to SEQ ID NO. 14. In some embodiments, the Fc domain comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 14. In some embodiments, the Fc domain comprises an amino acid sequence identical to SEQ ID NO. 14.

In some embodiments, the polypeptide comprising from the N-terminus to the C-terminus a formula selected from the group comprising of X₁-L₁-X₂ or X₂-L₁-X₁ comprises an amino acid sequence substantially similar to SEQ ID NO. 15: MIFLLLMLSLELQLHQTAALFTVTVPKELYIIEHGSNVTLECNFDTGSHVNLGAITASLQK VENDTSPHRERATLLEEQLPLGKASFHIPQVQVRDEGQYQCIIIYGVAWDYKYLTLKVK ASYRKINTHILKVPETDEVELTCQATGYPLAEVSWPNVSVPANTSHSRTPEGLYQVTSVL RLKPPPGRNFSCVFWNTHVRELTLASIDLQSQMEPRTHPTGSGSGGGSGSGGGSGSGSG LSHFCSGVIHVTKEVKEVATLSCGHNVSVEELAQTRIYWQKEKKMVLTMMSGDMNIW PEYKNRTIFDITNNLSIVILALRPSDEGTYECVVLKYEKDAFKREHLAEVTLSVKADFPTP SISDFEIPTSNIRRIICSTSGGFPEPHLSWLENGEELNAINTTVSQDPETELYAVSSKLDFNM TTNHSFMCLIKYGHLRVNQTFNWNTTKQEHFPD (SEQ ID NO. 15) or an active fragment thereof. In some embodiments, the polypeptide comprises an amino acid sequence having at least 60% identity to SEQ ID NO. 15. In some embodiments, the polypeptide comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 15. In some embodiments, the polypeptide comprises an amino acid sequence identical to SEQ ID NO. 15.

In some embodiments, the polypeptide comprising from the N-terminus to the C-terminus a formula selected from the group comprising of X₁-L₁-X₂ or X₂-L₁-X₁ comprises an amino acid sequence substantially similar to SEQ ID NO. 16: MRIFAVFIFMTYWHLLNAFTVTVPKDLYVVEYGSNMTIECKFPVEKQLDLAALIVYWE MEDKNIIQFVHGEEDLKVQHSSYRQRARLLKDQLSLGNAALQITDVKLQDAGVYRCMI SYGGADYKRITVKVNAPYNKINQRILVVDPVTSEHELTCQAEGYPKAEVIWTSSDHQVL SGKTTTTNSKREEKLFNVTSTLRINTTTNEIFYCTFRRLDPEENHTAELVIPELPLAHPPNE RGSGSGGGSGSGGGSGSGSAPLKIQAYFNETADLPCQFANSQNQSLSELVVFWQDQENL VLNEVYLGKEKFDSVHSKYMGRTSFDSDSWTLRLHNLQIKDKGLYQCIIHHKKPTGMIR IHQMNSELSVLANFSQPEIVPISNITENVYINLTCSSIHGYPEPKKMSVLLRTKNSTIEYDG VMQKSQDNVTELYDVSISLSVSFPDVTSNMTIFCILETDKTRLLSSPFSIELEDPQPPPDHIP (SEQ ID NO. 16) or an active fragment thereof. In some embodiments, the polypeptide comprises an amino acid sequence having at least 60% identity to SEQ ID NO. 16. In some embodiments, the polypeptide comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 16. In some embodiments, the polypeptide comprises an amino acid sequence identical to SEQ ID NO. 16.

In some embodiments, the polypeptide comprising from the N-terminus to the C-terminus a formula selected from the group comprising of LS-X₁-L₁-X₂-L₂-X₃, LS-X₁-L₁-X₃-L₂-X₂, LS-X₂-Lt₁-X₁-L₂-X₃, or LS-X₂-L₁-X₃-L₂-X₁ comprises an amino acid sequence substantially similar to SEQ ID NO. 17: MIFLLLMLSLELQLHQIAALFTVTVPKELYIIEHGSNVTLECNFDTGSHVNLGAITASLQK VENDTSPHRERATLLEEQLPLGKASFHIPQVQVRDEGQYQCIIIYGVAWDYKYLTLKVK ASYRKINTHILKVPETDEVELTCQATGYPLAEVSWPNVSVPANTSHSRTPEGLYQVTSVL RLKPPPGRNFSCVFWNTHVRELTLASIDLQSQMEPRTHPTGSGSGGGSGSGGGSGSGSG LSHFCSGVIHVTKEVKEVATLSCGHNVSVEELAQTRIYWQKEKKMVLTMMSGDMNIW PEYKNRTIFDITNNLSIVILALRPSDEGTYECVVLKYEKDAFKREHLAEVTLSVKADFPTP SISDFEIPTSNIRRIICSTSGGFPEPHLSWLENGEELNAINTTVSQDPETELYAVSSKLDFNM TTNHSFMCLIKYGHLRVNQTFNWNTTKQEHFPDDKTHTCPPCPAPELLGGPSVFLFPPKP KDTLMISRTPEVTCVVVDVSHEDPEVKFNWYVDGVEVHNAKTKPREEQYNSTYRVVS VLTVLHQDWLNGKEYKCKVSNKALPAPIEKTISKAKGQPREPQVYTLPPSRDELTKNQV SLTCLVKGFYPSDIAVEWESNGQPENNYKTTPPVLDSDGSFFLYSKLTVDKSRWQQGNV FSCSVMHEALHNHYTQKSLSLSPGK (SEQ ID NO. 17) or an active fragment thereof. In some embodiments, the polypeptide comprises an amino acid sequence having at least 60% identity to SEQ ID NO. 17. In some embodiments, the polypeptide comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 17. In some embodiments, the polypeptide comprises an amino acid sequence identical to SEQ ID NO. 17.

In some embodiments, the polypeptide comprising from the N-terminus to the C-terminus a formula selected from the group comprising of LS-X₁-L₁-X₂-L₂-X₃, LS-X₁-L₁-X₃-L₂-X₂, LS-X₂-L₁-X₁-L₂-X₃, or LS-X₂-L₁-X₃-L₂-X₁ comprises an amino acid sequence substantially similar to SEQ ID NO. 18: MRIFAVFIFMTYWHLLNAFTVTVPKDLYVVEYGSNMTIECKFPVEKQLDLAALIVYWE MEDKNIIQFVHGEEDLKVQHSSYRQRARLLKDQLSLGNAALQITDVKLQDAGVYRCMI SYGGADYKRITVKVNAPYNKINQRILVVDPVTSEHELTCQAEGYPKAEVIWTSSDHQVL SGKTTITNSKREEKLFNVTSTLRINTTTNEIFYCTFRRLDPEENHTAELVIPELPLAHPPNE RGSGSGGGSGSGGGSGSGSAPLKIQAYFNETADLPCQFANSQNQSLSELVVFWQDQENL VLNEVYLGKEKFDSVHSKYMGRTSFDSDSWTLRLHNLQIKDKGLYQCIIHHKKPTGMIR IHQMNSELSVLANFSQPEIVPISNITENVYINLTCSSIHGYPEPKKMSVLLRTKNSTIEYDG VMQKSQDNVTELYDVSISLSVSFPDVTSNMTIFCILETDKTRLLSSPFSIELEDPQPPPDHIP DKTHTCPPCPAPELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO. 18) or an active fragment thereof. In some embodiments, the polypeptide comprises an amino acid sequence having at least 60% identity to SEQ ID NO. 18. In some embodiments, the polypeptide comprises an amino acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 18. In some embodiments, the polypeptide comprises an amino acid sequence identical to SEQ ID NO. 18.

In some embodiments a nucleic acid molecule is provided. In some embodiments, the nucleic acid molecule encodes for a polypeptide as provided for herein. In some embodiments, the nucleic acid molecule encoding for the polypeptide comprises a nucleic acid sequence substantially similar to SEQ ID NO. 19: ATGATTTTTCTCTTGCTTATGCTTTCTCTTGAGTTGCAACTTCATCAGATTGCCGCCCT TTTTACCGTCACAGTCCCTAAAGAACTTTACATAATCGAGCACGGTAGCAACGTAAC GCTTGAATGTAACTTTGACACGGGGAGTCACGTCAATCTTGGAGCTATTACGGCGTC TCTTCAGAAGGTGGAAAACGATACGAGCCCTCACCGGGAGCGCGCTACCCTGCTTG AAGAACAGTTGCCGCTTGGAAAAGCATCCTTTCACATACCGCAGGTTCAAGTTCGAG ATGAAGGCCAATACCAATGCATAATTATTTATGGAGTCGCCTGGGACTATAAGTACC TGACATTGAAGGTTAAAGCGAGCTATAGGAAGATCAATACTCACATACTGAAGGTC CCGGAGACTGATGAAGTGGAATTGACTTGTCAGGCAACAGGGTATCCTTTGGCGGA GGTTAGTTGGCCTAATGTITCCGTGCCCGCGAACACATCTCATTCAAGGACACCAGA GGGCCTGTATCAGGTGACCTCCGTTCTGCGCCTGAAGCCACCGCCAGGTAGGAACTT TAGCTGCGTCTTCTGGAATACTCATGTTCGAGAACTCACACTTGCTTCAATCGACCTT CAGTCCCAGATGGAGCCTAGAACTCATCCTACCGGATCAGGTAGTGGCGGCGGAAG CGGGAGTGGCGGAGGCTCCGGGTCTGGTTCCGGCCTGTCACACTTTTGTTCTGGCGT AATCCACGTCACTAAGGAGGTTAAAGAGGTAGCCACCCTTTCTTGTGGTCATAATGT CTCTGTGGAAGAACTTGCTCAAACAAGAATCTACTGGCAAAAGGAGAAGAAAATGG TCTTGACGATGATGAGCGGTGACATGAACATATGGCCAGAGTACAAGAATCGCACC ATTTTCGACATAACCAACAATCTTTCAATCGTAATACTGGCCCTCAGGCCGTCTGAT GAAGGTACGTATGAGTGCGTTGTCTTGAAATACGAAAAAGACGCCTTCAAGCGAGA ACATCTGGCGGAGGTGACACTTTCTGTTAAAGCCGATTTCCCAACTCCTTCTATTTCA GATTTTGAAATCCCGACCTCTAACATCCGGAGAATTATCTGCAGCACGTCAGGGGGG TTTCCTGAACCACATTTGTCTTGGCTTGAGAATGGCGAAGAATTGAACGCCATCAAC ACCACCGTTAGTCAAGACCCAGAGACGGAACTGTATGCCGTTTCAAGTAAGCTCGAT TTTAATATGACAACTAACCACTCCTTCATGTGTCTGATAAAATATGGACATCTGCGG GTAAATCAGACATTTAACTGGAACACGACAAAACAAGAGCATTTCCCAGAT (SEQ ID NO. 19) or an active fragment thereof. In some embodiments, the nucleic acid molecule comprises a nucleic acid sequence having at least 60% identity to SEQ ID NO. 19. In some embodiments, the nucleic acid molecule comprises a nucleic acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 19. In some embodiments, the nucleic acid molecule comprises a nucleic acid sequence identical to SEQ ID NO. 19. The sequence of SEQ ID NO. 19 (as well as other nucleotide sequences provided for herein) is an exemplary sequence and is not meant to be limiting in any way. Due to the degenerate nature of codons, other nucleic acid molecules can be used. In some embodiments, the nucleic acid molecule is codon optimized for expression in a bacterial system. In some embodiments, the nucleic acid molecule is codon optimized for expression in a eukaryotic system or cell. In some embodiments, the nucleic acid molecule is a DNA or RNA molecule that encodes a polypeptide as provided for herein. In some embodiments, the RNA molecule is a mRNA molecule.

In some embodiments a nucleic acid molecule is provided. In some embodiments, the nucleic acid molecule encodes for a polypeptide as provided for herein. In some embodiments, the nucleic acid molecule encoding for the polypeptide comprises a nucleic acid sequence substantially similar to SEQ ID NO. 20: ATGCGCATATTCGCAGTCTTTATATTTATGACTTATTGGCATCTTCTTAATGCATTTA CTGTCACTGTTCCCAAGGATCTGTACGTTGTAGAGTACGGCTCCAACATGACTATAG AGTGCAAATFTCCAGTCGAGAAACAGCTCGACTTGGCAGCACTCATTGTGTACTGGG AAATGGA AGACAAAAACATCATTCAGTTCGTCCACGGGGAGGAGGACTTGAAAGTT CAACACTCATCCTATCGCCAACGAGCCCGCCTGTTGAAGGACCAGCTTTCTCTCGGT AACGCTGCACTTCAGATTACGGATGTTAAGTTGCAGGATGCAGGAGTCTACCGATGT ATGATTAGTTACGGCGGTGCCGATTACAAGAGGATCACTGTTAAAGTCAACGCTCCT TACAATAAAATAAACCAACGGATTCTCGTGGTAGATCCTGTGACTTCCGAGCACGAA TTGACGTGCCAAGCAGAAGGATACCCGAAAGCCGAGGTGATCTGGACAAGCTCCGA TCATCAAGTCCTCTCAGGGAAGACGACGACCACGAACTCTAAGAGGGAAGAGAAGC TGTTCAACGTAACATCAACGCTCCGCATTAACACGACTACTAATGAGATATTCTACT GCACTTTCCGGCGACTCGATCCGGAGGAAAACCATACAGCGGAGCTTGTGATTCCG GAACTTCCACTCGCGCATCCTCCGAACGAGCGGGGATCAGGTAGTGGCGGCGGATC CGGGAGTGGCGGGGGCAGCGGGTCTGGTTCCGCACCGTTGAAAATACAAGCGTACT TCAACGAAACTGCAGACCTTCCGTGTCAATTCGCGAACTCACAAAATCAATCCCTTA GTGAGCTTGTAGTATTTTGGCAGGACCAGGAAAATCTCGTCCTGAACGAAGTTTATC TCGGTAAAGAGAAGTTCGACAGCGTACATAGTAAATACATGGGCAGAACTTCTTTC GATAGTGATAGTTGGACGCTCAGGTTGCATAACCTCCAGATCAAGGATAAAGGCCT GTATCAATGTATTATCCACCACAAAAAACCCACAGGCATGATACGGATACATCAAA TGAACTCTGAACTGTCAGTCTTGGCAAATTTCAGTCAACCTGAGATTGTACCTATCA GCAACATTACCGAGAATGTCTACATTAATCTCACTTGCTCCAGCATACATGGGTATC CAGAACCTAAGAAAATGTCCGTACTCCTTAGAACGAAGAACTCAACTATTGAGTAC GACGGAGTAATGCAAAAATCCCAAGATAATGTCACAGAGCTTTATGATGTGAGTAT ATCACTCAGTGTATCATTCCCTGATGTGACTTCAAATATGACTATTTTCTGTATATTG GAGACAGATAAAACAAGGCTGTTGAGTAGTCCTTTCTCAATAGAGTTGGAGGACCC ACAACCGCCACCCGACCACATCCCC (SEQ ID NO. 20) or an active fragment thereof. In some embodiments, the nucleic acid molecule comprises a nucleic acid sequence having at least 60% identity to SEQ ID NO. 20. In some embodiments, the nucleic acid molecule comprises a nucleic acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 20. In some embodiments, the nucleic acid molecule comprises a nucleic acid sequence identical to SEQ ID NO. 20. The sequence of SEQ ID NO. 20 is an exemplary sequence and is not meant to be limiting in any way. Due to the degenerate nature of codons, other nucleic acid molecules can be used. In some embodiments, the nucleic acid molecule is codon optimized for expression in a bacterial system. In some embodiments, the nucleic acid molecule is codon optimized for expression in a eukaryotic system or cell. In some embodiments, the nucleic acid molecule is a DNA or RNA molecule that encodes a polypeptide as provided for herein. In some embodiments, the RNA molecule is a mRNA molecule.

In some embodiments a nucleic acid molecule is provided. In some embodiments, the nucleic acid molecule encodes for a polypeptide as provided for herein. In some embodiments, the nucleic acid molecule encoding for the polypeptide comprises a nucleic acid sequence substantially similar to SEQ ID NO. 21. ATGATTTCTCTTGCTTATGCTTTCTCTTGAGTTGCAACTTCATCAGATTGCCGCCCT TTTTTACCGTCACAGTCCCTAAAGAACTTTACATAATCGAGCACGGTAGCAACGTAAC GCTTGAATGTAACTTTGACACGGGGAGTCACGTCAATCTTGGAGCTATTACGGCGTC TCTTCAGAAGGTGGAAAACGATACGAGCCCTCACCGGGAGCGCGCTACCCTGCTTG AAGAACAGTTGCCGCTTGGAAAAGCATCCTTTCACATACCGCAGGTTCAAGTTCGAG ATGAAGGCCAATACCAATGCATAATTATTTATGGAGTCGCCTGGGACTATAAGTACC TGACATTGAAGGTTAAAGCGAGCTATAGGAAGATCAATACTCACATACTGAAGGTC CCGGAGACTGATGAAGTGGAATTGACTTGTCAGGCAACAGGGTATCCTTTGGCGGA GGTTAGTTGGCCTAATGTTTCCGTGCCCGCGAACACATCTCATTCAAGGACACCAGA GGGCCTGTATCAGGTGACCTCCGTTCTGCGCCTGAAGCCACCGCCAGGTAGGAACTT TAGCTGCGTCTTCTGGAATACTCATGTTCGAGAACTCACACTTGCTTCAATCGACCTT CAGTCCCAGATGGAGCCTAGAACTCATCCTACCGGATCAGGTAGTGGCGGCGGAAG CGGGAGTGGCGGAGGCTCCGGGTCTGGTTCCGGCCTGTCACACTTTTGTTCTGGCGT AATCCACGTCACTAAGGAGGTTAAAGAGGTAGCCACCCTTTCTTGTGGTCATAATGT CTCTGTGGAAGAACTTGCTCAAACAAGAATCTACTGGCAAAAGGAGAAGAAAATGG TCTTGACGATGATGAGCGGTGACATGAACATATGGCCAGAGTACAAGAATCGCACC ATTTTCGACATAACCAACAATCTTTCAATCGTAATACTGGCCCTCAGGCCGTCTGAT GAAGGTACGTATGAGTGCGTTGTCTTGAAATACGAAAAAGACGCCTTCAAGCGAGA ACATCTGGCGGAGGTGACACTTTCTGTTAAAGCCGATTTCCCAACTCCTTCTATTTCA GATTTTGAAATCCCGACCTCTAACATCCGGAGAATTATCTGCAGCACGTCAGGGGGG TTTCCTGAACCACATTTGTCTTGGCTTGAGAATGGCGAAGAATTGAACGCCATCAAC ACCACCGTTAGTCAAGACCCAGAGACGGAACTGTATGCCGTTTCAAGTAAGCTCGAT TTTAATATGACAACTAACCACTCCTTCATGTGTCTGATAAAATATGGACATCTGCGG GTAAATCAGACATTTAACTGGAACACGACAAAACAAGAGCATTTCCCAGATGATAA AACCCATACCTGTCCTCCTTGCCCTGCCCCCGAACTGCTGGGTGGGCCTAGCGTGTT CCTTTTCCCGCCCAAGCCGAAGGACACACTCATGATCAGTCGAACGCCCGAAGTTAC GTGCGTGGTCGTAGATGTGTCCCACGAAGATCCAGAGGTGAAGTTTAATTGGTACGT TGATGGTGTAGAGGTACATAATGCCAAAACAAAGCCACGCGAAGAGCAGTATAATA GCACCTATAGAGTAGTTAGCGTTCTGACAGTTCTCCACCAGGATTGGCTTAACGGCA AAGAGTACAAGTGTAAAGTATCAAACAAAGCACTTCCTGCACCCATTGAAAAAACT ATATCTAAGGCCAAAGGCCAACCTAGGGAACCGCAGGTCTACACGCTGCCCCCGAG CAGAGACGAGCTTACGAAGAACCAAGTCTCACTGACGTGTCTTGTCAAGGGTITITA CCCCAGTGACATTGCAGTAGAGTGGGAATCAAATGGGCAACCAGAAAACAACTATA AAACTACACCACCAGTCTTGGACAGTGATGGATCATTCTTCTTGTATTCAAAACTTA CGGTTGACAAGAGCCGCTGGCAACAGGGCAACGTGTTCAGCTGCTCCGTGATGCAT GAGGCGCTTCACAACCATTACACCCAAAAGAGTCTTTCACTCTCTCCCGGAAAA (SEQ ID NO. 21) or an active fragment thereof. In some embodiments, the nucleic acid molecule comprises a nucleic acid sequence having at least 60% identity to SEQ ID NO. 21. In some embodiments, the nucleic acid molecule comprises a nucleic acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 21. In some embodiments, the nucleic acid molecule comprises a nucleic acid sequence identical to SEQ ID NO. 21. The sequence of SEQ ID NO. 21 is an exemplary sequence and is not meant to be limiting in any way. Due to the degenerate nature of codons, other nucleic acid molecules can be used. In some embodiments, the nucleic acid molecule is codon optimized for expression in a bacterial system. In some embodiments, the nucleic acid molecule is codon optimized for expression in a eukaryotic system or cell. In some embodiments, the nucleic acid molecule is a DNA or RNA molecule that encodes a polypeptide as provided for herein. In some embodiments, the RNA molecule is a mRNA molecule.

In some embodiments a nucleic acid molecule is provided. In some embodiments, the nucleic acid molecule encodes for a polypeptide as provided for herein. In some embodiments, the nucleic acid molecule encoding for the polypeptide comprises a nucleic acid sequence substantially similar to SEQ ID NO. 22: ATGCGCATATTCGCAGTCTTTATATTTATGACTTATTGGCATCTTCTTAATGCATTTA CTGTCACTGTTCCCAAGGATCTGTACGTTGTAGAGTACGGCTCCAACATGACTATAG AGTGCAAATITCCAGTCGAGAAACAGCTCGACTTGGCAGCACTCATTGTGTACTGGG AAATGGAAGACAAAAACATCATTCAGTTCGTCCACGGGGAGGAGGACTTGAAAGTT CAACACTCATCCTATCGCCAACGAGCCCGCCTGTTGAAGGACCAGCTTTCTCTCGGT AACGCTGCACTTCAGATTACGGATGTTAAGTTGCAGGATGCAGGAGTCTACCGATGT ATGATTAGTTACGGCGGTGCCGATTACAAGAGGATCACTGTTAAAGTCAACGCTCCT TACAATAAAATAAACCAACGGATTCTCGTGGTAGATCCTGTGACTTCCGAGCACGAA TTGACGTGCCAAGCAGAAGGATACCCGAAAGCCGAGGTGATCTGGACAAGCTCCGA TCATCAAGTCCTCTCAGGGAAGACGACGACCACGAACTCTAAGAGGGAAGAGAAGC TGTTCAACGTAACATCAACGCTCCGCATTAACACGACTACTAATGAGATATTCTACT GCACTTTCCGGCGACTCGATCCGGAGGAAAACCATACAGCGGAGCTTGTGATTCCG GAACTTCCACTCGCGCATCCTCCGAACGAGCGGGGATCAGGTAGTGGCGGCGGATC CGGGAGTGGCGGGGGCAGCGGGTCTGGTTCCGCACCGTTGAAAATACAAGCGTACT TCAACGAAACTGCAGACCTTCCGTGTCAATTCGCGAACTCACAAAATCAATCCCTTA GTGAGCTTGTAGTATTTTGGCAGGACCAGGAAAATCTCGTCCTGAACGAAGTTTATC TCGGTAAAGAGAAGTTCGACAGCGTACATAGTAAATACATGGGCAGAACTTCTTTC GATAGTGATAGTTGGACGCTCAGGTTGCATAACCTCCAGATCAAGGATAAAGGCCT GTATCAATGTATTATCCACCACAAAAAACCCACAGGCATGATACGGATACATCAAA TGAACTCTGAACTGTCAGTCTTGGCAAATTTCAGTCAACCTGAGATTGTACCTATCA GCAACATTACCGAGAATGTCTACATTAATCTCACTTGCTCCAGCATACATGGGTATC CAGAACCTAAGAAAATGTCCGTACTCCTTAGAACGAAGAACTCAACTATTGAGTAC GACGGAGTAATGCAAAAATCCCAAGATAATGTCACAGAGCTTTATGATGTGAGTAT ATCACTCAGTGTATCATTCCCTGATGTGACTTCAAATATGACTATTTTCTGTATATTG GAGACAGATAAAACAAGGCTGTTGAGTAGTCCTTTCTCAATAGAGTTGGAGGACCC ACAACCGCCACCCGACCACATCCCCGATAAAACCCATACCTGTCCTCCTTGCCCTGC CCCCGAACTGCTGGGTGGGCCTAGCGTGTTCCTTTTCCCGCCCAAGCCGAAGGACAC ACTCATGATCAGTCGAACGCCCGAAGTTACGTGCGTGGTCGTAGATGTGTCCCACGA AGATCCAGAGGTGAAGTTTAATTGGTACGTTGATGGTGTAGAGGTACATAATGCCA AAACAAAGCCACGCGAAGAGCAGTATAATAGCACCTATAGAGTAGTTAGCGTTCTG ACAGTTCTCCACCAGGATTGGCTTAACGGCAAAGAGTACAAGTGTAAAGTATCAAA CAAAGCACTTCCTGCACCCATTGAAAAAACTATATCTAAGGCCAAAGGCCAACCTA GGGAACCGCAGGTCTACACGCTGCCCCCGAGCAGAGACGAGCTTACGAAGAACCAA GTCTCACTGACGTGTCTTGTCAAGGGITrTIACCCCAGTGACATTGCAGTAGAGTGG GAATCAAATGGGCAACCAGAAAACAACTATAAAACTACACCACCAGTCTTGGACAG TGATGGATCATTCTTCTTGTATTCAAAACTTACGGTTGACAAGAGCCGCTGGCAACA GGGCAACGTGTTCAGCTGCTCCGTGATGCATGAGGCGCTTCACAACCATTACACCCA AAAGAGTCTTTCACTCTCTCCCGGAAAA (SEQ ID NO. 22) or an active fragment thereof. In some embodiments, the nucleic acid molecule comprises a nucleic acid sequence having at least 60%, identity to SEQ ID NO. 22. In some embodiments, the nucleic acid molecule comprises a nucleic acid sequence having at least 65%, 70%, 75%, 80%, 85%, 86%, 87%, 88%, 89%, 90%, 91%, 92%, 93%, 94%, 95%, 96%, 97%, 98%, or 99% identity to SEQ ID NO. 22. In some embodiments, the nucleic acid molecule comprises a nucleic acid sequence identical to SEQ ID NO. 22. The sequence of SEQ ID NO. 22 is an exemplary sequence and is not meant to be limiting in any way. Due to the degenerate nature of codons, other nucleic acid molecules can be used. In some embodiments, the nucleic acid molecule is codon optimized for expression in a bacterial system. In some embodiments, the nucleic acid molecule is codon optimized for expression in a eukaryotic system or cell. In some embodiments, the nucleic acid molecule is a DNA or RNA molecule that encodes a polypeptide as provided for herein. In some embodiments, the RNA molecule is a mRNA molecule.

In some embodiments, a vector is provided comprising a nucleic acid molecule as provided for herein. In some embodiments, the vector is a plasmid. In some embodiments, the vector is a virus. In some embodiments, a plasmid is provided comprising a nucleic acid molecule as provided for herein. In some embodiments, a composition is provided comprising an encapsulated nucleic acid molecule as provided for herein.

In some embodiments, a virus is provided. In some embodiments, the virus is a recombinant virus. In some embodiments, the recombinant virus is selected from the group consisting of lentivirus, adenovirus, adeno-associated virus, or the like. In some embodiments, the recombinant virus is a recombinant adenovirus. In some embodiments, the recombinant adenovirus is replication competent. In some embodiments, the recombinant adenovirus is replication incompetent. In some embodiments, the replication-incompetent recombinant virus further comprises a defective or modified E1 gene, E3 gene, E4 gene, E4 promoter, hexon gene, penton gene, fiber gene, or combination thereof. In some embodiments, the replication-incompetent recombinant virus comprises a defective or modified E1 gene.

In some embodiments, the recombinant virus comprises a nucleic acid molecule as provided for herein. In some embodiments, the nucleic acid molecule is selected from the group consisting of SEQ ID NO. 19-SEQ ID NO. 22. In some embodiments, the recombinant virus comprises a nucleic acid molecule encoding for a polypeptide as provided for herein. In some embodiments, the polypeptide is selected from the group consisting of SEQ ID NO. 15-SEQ ID NO. 18. In some embodiments, the nucleic acid molecule is flanked by a 5′ adenoviral ITR and a 3′ adenoviral ITR.

In some embodiments, a cell is provided. In some embodiments, the cell comprises a polypeptide molecule as provided for herein. In some embodiments, the polypeptide molecule is selected from the group consisting of SEQ ID NO. 15-SEQ ID NO. 18. In some embodiments, the cell comprises a nucleic acid molecule encoding for a polypeptide as provided for herein. In some embodiments the nucleic acid molecule is selected from the group consisting of SEQ ID NO. 19-SEQ ID NO. 22.

In some embodiments, the cell further comprises a chimeric antigen receptor (“CAR”). CARs can be used to treat cancer or tumors in a subject. In some embodiments, the activity of the CAR can be enhanced by co-expressing a polypeptide as provided for herein with the CAR. Thus, in some embodiments, a cell is provided comprising a CAR and a polypeptide as provided for herein.

The cell can be any type of suitable cell. In some embodiments, the cell is an immune cell, such as, but not limited to a T-cell, a NK cell, a dendritic cell, a monocyte, a B-cell, a myeloid cell, and the like.

In some embodiments, a host cell is provided. In some embodiments, the host cell contains a competent E1 gene, E3 gene, E4 gene, E4 promoter, hexon gene, penton gene, fiber gene or a combination thereof to complement any defective or modified gene in the recombinant virus. In some embodiments, the host cell contains a competent E1 gene to complement the defective or modified E1 gene in the recombinant virus. In some embodiments, the competent E1 gene, E3 gene, E4 gene, E4 promoter, hexon gene, penton gene, fiber gene or a combination thereof are provided to the host cell via contacting the host cell transiently. In some embodiments, the host cell contains, within its genome, the competent E1 gene, E3 gene, E4 gene, E4 promoter, hexon gene, penton gene, fiber gene or a combination thereof.

### Pharmaceutical Compositions

In another aspect, the present embodiments provide compositions, e.g., pharmaceutically acceptable compositions, which include a polypeptide as provided for herein or a nucleic acid molecule encoding the same, which can be, for example, be formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, local, topical, spinal or epidermal administration (e.g., by injection or infusion). In some embodiments, suitable pharmaceutically acceptable carries include, but are not limited to, water, silicone, waxes, petroleum jelly, polyethylene glycol, propylene glycol, liposomes, cationic lipids such as 1,2,-dioleoyl-3-trimethylammonium propane (DOTAP), 1,2,-dioleoyl-sn-glycero-3-phosphochiline (DOPC), and 1,2-dioleoyl-sn-glycero-3-phosphoethanolamine (DOPE), sugars such as mannitol and lactose, and other materials depending on the specific type of formulation used. In some embodiments, suitable pharmaceutically acceptable carries include, but are not limited to, nanoparticles such as gold or metallic nanoparticles.

In some embodiments, the lipids and liposomes comprises a cationic lipid, such as, but not limited to, 1,2-Dioleoyl-3-Trimethylammonium-Propane (DOTAP), 1,2,-dioleoyl-sn-glycero-3-phosphochiline (DOPC), 1,2-dioleoyl-sn-glycero-3-phosphoethanolamine (DOPE), N-[1-(2,3-dioleyloxy)propyl]-N,N,N-trimethylammonium chloride (DOTMA), 5-carboxyspermylglycinedioctadecylamide (DOGS), 2,3-dioleyloxy-N-[2(spermine-carboxamido)ethyl]-N,N-dimethyl-1-propanamin-ium (DOSPA), 1,2-Dioleoyl-3-Dimethylammonium-Propane (DODAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), heptatriaconta-6,9,28,31-tetraen19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dilinolenyloxy-N,N-dimethyl-3-aminopropane (DLenDMA), N-dioleyl-N,N-dimethyl ammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis, cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminoethyl-[1,3]-dioxolane (DLin-K-XTC2-DMA), or C12-200.

In some embodiments, the pharmaceutical composition comprises a vector comprising a nucleic acid molecule encoding a polypeptide as provided for herein. In some embodiments, the nucleic acid molecule is a DNA molecule or an RNA molecule. In some embodiments, the vector is a virus, such as those provided for herein. In some embodiments, the pharmaceutical composition comprises a polypeptide as provided for herein.

The compositions may be in a variety of forms. These include, for example, liquid, semi-solid and solid dosage forms, such as liquid solutions (e.g., injectable and infusible solutions), dispersions or suspensions, liposomes and suppositories. The form depends on the intended mode of administration and therapeutic application. Typical compositions are in the form of injectable or infusible solutions. In an embodiment the mode of administration is parenteral (e.g., intravenous, subcutaneous, intraperitoneal, intramuscular). In an embodiment, the therapeutic molecule is administered by intravenous infusion or injection. In some embodiments, the therapeutic molecule is administered by intramuscular or subcutaneous injection. In some embodiments, the therapeutic molecule is administered locally, e.g., by injection, or topical application, to a target site. For example, the pharmaceutical compositions can be lyophilized and reconstituted for use prior to administration to the patient.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intravesicular, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation can be vacuum drying and freeze-drying that yields a powder of the active ingredient plus any additional desired ingredient from a previously sterile-filtered solution thereof. The proper fluidity of a solution can be maintained, for example, using a coating such as lecithin, by the maintenance of the required particle size in the case of dispersion, and using surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In some embodiments, the pharmaceutical composition can be administered intranasally or to the mucosa, including but not limited to be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer composition by other than parenteral administration, it may be necessary to coat the composition with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms is dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks, or, in some embodiments, the dosing schedule can be, once every month, every 2 months, every 3 months, or every 6 months. In one embodiment, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, the infusion rate of about 110 to 130 mg/m2 achieves a level of about 3 mg/kg. In other embodiments, the therapeutic compound can be administered by intravenous infusion at a rate of less than 10 mg/min, e.g., less than or equal to 5 mg/min to reach a dose of about 1 to 100 mg/m2, e.g., about 5 to 50 mg/m2, about 7 to 25 mg/m2, or, about 10 mg/m2. In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of the compositions, polypeptides, or nucleic acid molecules encoding the same. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule t is outweighed by the therapeutically beneficial effects. A “therapeutically effective dosage” preferably inhibits a measurable parameter, e.g., tumor growth, by at least about 20%, by at least about 40%, by at least about 60%, and by at least about 80% relative to untreated subjects. The ability of a compound to inhibit a measurable parameter, e.g., tumor growth, can be evaluated in an animal model system predictive of efficacy in tumor growth. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount can be less than the therapeutically effective amount.

Also provided herein are kits comprising compositions as described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the therapeutic molecule for administration; pharmaceutically acceptable carriers; and devices or other materials for administration to a subject.

### Combinations

The compositions provided herein can also be administered in conjunction with other agents useful for treating the condition with which the patient is suffering from. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in which compositions provided for herein are administered at least once during a course of treatment that involves administering at least one other therapeutic agent to the patient.

### Methods

In some embodiments, methods of preparing and using the compositions described herein are provided.

In some embodiments, a method for producing a cell as provided for herein is provided. In some embodiments, the method comprises contacting the cell with a vector comprising a nucleic acid molecule encoding for a polypeptide as provided for herein. In some embodiments, the nucleic acid molecule is any nucleic acid molecule as provided for herein. In some embodiments, the nucleic acid molecule is selected from the group comprising SEQ ID NO. 19-SEQ ID NO. 22. In some embodiments, the nucleic acid molecule encodes for any polypeptide as provided for herein. In some embodiments, the nucleic acid molecule encodes for a polypeptide selected from the group comprising SEQ ID NO. 15-SEQ ID NO. 18.

In some embodiments, the vector of the method is a plasmid or virus. In some embodiments, the virus is an adenovirus or a lentivirus. In some embodiments, the adenovirus is replication-incompetent or replication competent. In some embodiments the replication-incompetent recombinant virus further comprises a defective or modified E1 gene, E3 gene, E4 gene, E4 promoter, hexon gene, penton gene, fiber gene, or combination thereof. In some embodiments, the replication-incompetent recombinant virus comprises a defective or modified E1 gene. In some embodiments, the cell contains a competent E1 gene, E3 gene, E4 gene, E4 promoter, hexon gene, penton gene, fiber gene or a combination thereof to complement any defective or modified gene in the recombinant virus. In some embodiments, the cell contains a competent E1 gene to complement the defective or modified E1 gene in the recombinant virus. In some embodiments, the competent E1 gene, E3 gene, E4 gene, E4 promoter, hexon gene, penton gene, fiber gene or a combination thereof are provided to the cell via contacting the cell transiently. In some embodiments, the cell contains, within its genome, the competent E1 gene, E3 gene, E4 gene, E4 promoter, hexon gene, penton gene, fiber gene or a combination thereof.

In some embodiments, the contacting of the method comprises transducing or transfecting the cell with a vector. In some embodiments, the contacting of the method can be any known method of expressing a polypeptide of interest in a cell.

In some embodiments, the vector of the method further comprises a nucleic acid molecule encoding for at least a second heterologous molecule of interest. In some embodiments, the heterologous molecule of interest is selected from the group comprising a chimeric antigen receptor or an antigen. In some embodiments, the antigen is any substance that generates an immune response. In some embodiments, the antigen is a tumor antigen. In some embodiments, the antigen is a viral antigen. In some embodiments, the antigen is a bacterial or microbial antigen. In some embodiments, the vector further comprises a nucleic acid molecule encoding for a chimeric antigen receptor or a tumor antigen. In some embodiments, the vector further comprises a nucleic acid molecule encoding for a chimeric antigen receptor and an antigen as provided for herein. In some embodiments, the vector further comprises a nucleic acid molecule encoding for a chimeric antigen receptor and a tumor antigen.

In some embodiments, a method for producing a polypeptide as provided for herein is provided. In some embodiments, the method comprises transducing or transfecting a cell with a vector comprising a nucleic acid molecule encoding for the polypeptide under conditions sufficient to make the polypeptide. In some embodiments, the nucleic acid molecule is selected from the group comprising SEQ ID NO. 19-SEQ ID NO. 22. In some embodiments, the nucleic acid molecule encodes for any polypeptide as provided for herein. In some embodiments, the nucleic acid molecule encodes for a polypeptide selected from the group comprising SEQ ID NO. 15-SEQ ID NO. 18.

In some embodiments, the cell is transduced or transfected in vivo in a subject. In some embodiments, the cell is transduced or transfected ex vivo.

In some embodiments, the cell is a bacteria or mammalian cell, and the polypeptide is produced in order to obtain a recombinant polypeptide. The skilled artisan would understand the steps and protocols needed to produce and purify the polypeptide to produce a recombinant polypeptide. Such methods are within the scope of the present application.

In some embodiments, the cell can be any type of suitable cell. In some embodiments, the cell is an immune cell, such as, but not limited to a T-cell, a NK cell, a dendritic cell, a monocyte, a B-cell, a myeloid cell, and the like.

In some embodiments, the vector of the method further comprises a nucleic acid molecule encoding for at least a second heterologous molecule of interest. In some embodiments, the heterologous molecule of interest is selected from the group comprising a chimeric antigen receptor or an antigen. In some embodiments, the antigen is any substance that generates an immune response. In some embodiments, the antigen is a tumor antigen. In some embodiments, the antigen is a viral antigen. In some embodiments, the antigen is a bacterial or microbial antigen. In some embodiments, the vector further comprises a nucleic acid molecule encoding for a chimeric antigen receptor or a tumor antigen. In some embodiments, the vector further comprises a nucleic acid molecule encoding for a chimeric antigen receptor and an antigen as provided for herein. In some embodiments, the vector further comprises a nucleic acid molecule encoding for a chimeric antigen receptor and a tumor antigen.

In some embodiments, the vector of the method is a plasmid or virus. In some embodiments, the virus is an adenovirus or a lentivirus, such as a pseudotyped adenovirus or pseudotyped lentivirus. In some embodiments, the adenovirus is replication competent. In some embodiments, the adenovirus is replication incompetent. In some embodiments the replication-incompetent recombinant virus further comprises a defective or modified E1 gene, E3 gene, E4 gene, E4 promoter, hexon gene, penton gene, fiber gene, or combination thereof. In some embodiments, the cell contains a competent E1 gene, E3 gene, E4 gene, E4 promoter, hexon gene, penton gene, fiber gene or a combination thereof to complement any defective or modified gene in the recombinant virus. In some embodiments, the competent E1 gene, E3 gene, E4 gene, E4 promoter, hexon gene, penton gene, fiber gene or a combination thereof are provided to the cell via contacting the cell transiently. In some embodiments, the cell contains, within its genome, the competent E1 gene, E3 gene, E4 gene, E4 promoter, hexon gene, penton gene, fiber gene or a combination thereof.

In some embodiments, a method for modulating an immune response in a patient or subject in need thereof is provided. In some embodiments, the method comprises administering to the patient or subject a pharmaceutical composition comprising a polypeptide as provided for herein. In some embodiments, the polypeptide is selected from the group comprising polypeptide selected from the group comprising SEQ ID NO. 15-SEQ ID NO. 18. In some embodiments, the method comprises administering to the patient or subject a vector comprising a nucleic acid molecule as provided for herein. In some embodiments, the nucleic acid molecule is selected from the group comprising SEQ ID NO. 19-SEQ ID NO. 22. In some embodiments, the nucleic acid molecule encodes for any polypeptide as provided for herein. In some embodiments, the nucleic acid molecule encodes for a polypeptide selected from the group comprising SEQ ID NO. 15-SEQ ID NO. 18.

In some embodiments, the vector of the method is a plasmid or virus. In some embodiments, the virus is an adenovirus or a lentivirus, such as a pseudotyped adenovirus or pseudotyped lentivirus. In some embodiments, the adenovirus is replication competent. In some embodiments, the adenovirus is replication incompetent.

In some embodiments, the polypeptide enhances T-cell activation. In some embodiments, expression of the polypeptide enhances T-cell activation.

In some embodiments, the vector of the method further comprises a nucleic acid molecule encoding for at least a second heterologous molecule of interest. In some embodiments, the heterologous molecule of interest is selected from the group comprising a chimeric antigen receptor or an antigen. In some embodiments, the antigen is any substance that generates an immune response. In some embodiments, the antigen is a tumor antigen. In some embodiments, the antigen is a viral antigen. In some embodiments, the antigen is a bacterial or microbial antigen. In some embodiments, the vector further comprises a nucleic acid molecule encoding for a chimeric antigen receptor or a tumor antigen. In some embodiments, the vector further comprises a nucleic acid molecule encoding for a chimeric antigen receptor and an antigen as provided for herein. In some embodiments, the vector further comprises a nucleic acid molecule encoding for a chimeric antigen receptor and a tumor antigen.

In some embodiments, a method of treating cancer in a subject in need thereof is provided. In some embodiments, the method comprises administering to the subject a pharmaceutical composition comprising a polypeptide as provided for herein. In some embodiments, the polypeptide is selected from the group comprising polypeptide selected from the group comprising SEQ ID NO. 15-SEQ ID NO. 18. In some embodiments, the method comprises administering to the subject a vector comprising a nucleic acid molecule as provided for herein. In some embodiments, the nucleic acid molecule is selected from the group comprising SEQ ID NO. 19-SEQ ID NO. 22. In some embodiments, the nucleic acid molecule encodes for any polypeptide as provided for herein. In some embodiments, the nucleic acid molecule encodes for a polypeptide selected from the group comprising SEQ ID NO. 19-SEQ ID NO. 22.

In some embodiments, the cancer can be any cancer. In some embodiments, the cancer is lymphoma, leukemia, nasopharyngeal, gastric, cervical, hepatocellular, polyoma, anal, head and neck tumor. In some embodiments, the tumor is a lung cancer tumor. In some embodiments, the tumor is benign and metastatic forms of cancer, for example, ovarian cancer (e.g. ovarian carcinoma), reproductive cancers (breast, cervical, testicular, uterine, and placental cancers), lung cancer, gastric cancer, hepatic cancer, pancreatic cancer, bile duct cancer, cancer of the urinary bladder, kidney cancer, colon cancer, small bowel cancer, skin cancer, brain cancer, head and neck cancer, sarcoma, and germ cell tumors, among others. The previous recitation of cancers is not meant to be limiting in any way. The embodiments provided for herein may be used to treat any number of additional cancers. Any cancer wherein treatment with the embodiments provided for herein results in treatment of the cancer (as defined herein) is within the scope of the present application.

In some embodiments, the vector of the method is a plasmid or virus. In some embodiments, the virus is an adenovirus or a lentivirus, such as a pseudotyped adenovirus or pseudotyped lentivirus. In some embodiments, the adenovirus is replication competent. In some embodiments, the adenovirus is replication incompetent.

In some embodiments, the method of treating cancer further comprises the use of at least one additional therapeutic. In some embodiments, the additional therapeutic is an anti-cancer therapeutic. In some embodiments, the anti-cancer therapeutic is selected from the group including, but not limited to, chemotherapy, radiation therapy, targeted therapy, immunotherapy, stem cell or bone marrow transplant therapy, hormone therapy, photodynamic therapy, and the like, or any combination thereof.

Subjects undergoing chemotherapy or other anti-cancer therapies are often at an increased risk of infection from various sources. As such, in some embodiments, the at least one additional therapeutic is an anti-infective therapeutic. These can include but are not limited to anti-bacterial, anti-microbial, anti-viral, and the like. Anti-infective agents are well known in the art, and use of such anti-infective agents in combination with the methods recited are within the scope of this application. In some embodiments, the method of treating cancer further comprises the use of at least one additional anti-cancer therapeutic as provided for herein and at least one anti-infective therapeutic as provided for herein.

In some embodiments, a method for treating a disease or disorder in a subject in need thereof is provided. In some embodiments, the disease or disorder is selected from the group including, but not limited to, a viral infection, a bacterial infection, a fungal infection, or the like. In some embodiments, the method comprises administering to the to the subject a pharmaceutical composition comprising a polypeptide as provided for herein. In some embodiments, the polypeptide is selected from the group comprising polypeptide selected from the group comprising SEQ ID NO. 15-SEQ ID NO. 18. In some embodiments, the method comprises administering to the subject a vector comprising a nucleic acid molecule as provided for herein. In some embodiments, the nucleic acid molecule is selected from the group comprising SEQ ID NO. 19-SEQ ID NO. 22. In some embodiments, the nucleic acid molecule encodes for any polypeptide as provided for herein. In some embodiments, the nucleic acid molecule encodes for a polypeptide selected from the group comprising SEQ ID NO. 15-SEQ ID NO. 18.

In some embodiments, the vector of the method is a plasmid or virus. In some embodiments, the virus is an adenovirus or a lentivirus, such as a pseudotyped lentivirus. In some embodiments, the adenovirus is replication competent. In some embodiments, the adenovirus is replication incompetent.

### Enumerated Embodiments

In some embodiments, the following embodiments are provided:

1. A polypeptide comprising from the N-terminus to the C-terminus a formula of X₁-L₁-X₂ or a formula of X₂-L₁-X₁ wherein:
X₁ is an immune checkpoint polypeptide, or an active fragment thereof, such as, but not limited to, a CTLA-4 polypeptide, a PD-1 polypeptide, a PD-L1 polypeptide, a PD-L2 polypeptide, a TIM3 polypeptide, a LAG3 polypeptide, a VISTA polypeptide, a SINGLEC7 polypeptide, a SINGLEC9 polypeptide, a TIGIT polypeptide, a CD96 polypeptide, a BTLA polypeptide, a B7H3 polypeptide, a B7H4 polypeptide, a CD155 polypeptide, a HHLA2 polypeptide, a BTN3A1 polypeptide, or an active fragment thereof;
X₂ is a B7.1 polypeptide, or a B7.2 polypeptide; and
L₁ is a polypeptide linker.
   2. The polypeptide of embodiment 1, wherein the polypeptide comprising from the N-terminus to the C-terminus a formula of X₁-L₁-X₂.
   3. The polypeptide of embodiment 1, wherein the polypeptide comprising from the N-terminus to the C-terminus a formula of X₂-L₁-X₁.
   4. The polypeptide of any one of embodiments 1-3, wherein:
X₁ is a PD-L1 polypeptide; and
X₂ is a B7.1 polypeptide.
   5. The polypeptide of any one of embodiments 1-3, wherein:
X₁ is a PD-L1 polypeptide; and
X₂ is a B7.2 polypeptide.
   6. The polypeptide of any one of embodiments 1-3, wherein:
X₁ is a PD-L2 polypeptide; and
X₂ is a B7.1 polypeptide.
   7. The polypeptide of any one of embodiments 1-3, wherein:
X₁ is a PD-L2 polypeptide; and
X₂ is a B7.2 polypeptide.
   8. The polypeptide of any one of embodiments 1-7, wherein the PD-L1 polypeptide comprises a PD-L1 extracellular domain.
   9. The polypeptide of embodiment 10, wherein the PD-L1 polypeptide comprises an amino acid sequence having at least 85%, 86%, 97%, 88%, 89%, 90%, 91%, 92%, 93%, 94%, 95%, 96%, 97%, 98%, or 99%, identity to SEQ ID NO 2 or comprises the sequence of SEQ ID NO 2.
   10. The polypeptide of any one of embodiments 1-7, wherein the PD-L2 polypeptide comprises a PD-L2 extracellular domain.
   11. The polypeptide of embodiment 10, wherein the PD-L2 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 4.
   12. The polypeptide of embodiment 11, wherein the PD-L2 polypeptide comprises of SEQ ID NO 4.
   13. The polypeptide of any one of embodiments 1-12, wherein the B7.1 polypeptide comprises a B7.1 extracellular domain.
   14. The polypeptide of embodiment 13, wherein the B7.1 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 6 or comprises the sequence of SEQ ID NO 6.
   15. The polypeptide of any one of embodiments 1-12, wherein the B7.2 polypeptide comprises a B7.2 extracellular domain.
   16. The polypeptide of embodiment 15, wherein the B7.2 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 8 or comprises the sequence of SEQ ID NO 8.
   17. The polypeptide of any one of embodiments 1-16, wherein the polypeptide comprises a leader sequence.
   18. The polypeptide of embodiment 17, wherein the leader sequence is at the N-terminus of the polypeptide.
   19. The polypeptide of embodiment 17 or 18, wherein the leader sequence is a cell membrane targeting polypeptide.
   20. The polypeptide of any one of embodiments 17-19, wherein the leader sequence is a sequence of SEQ ID NO. 10, SEQ ID NO. 11, SEQ ID NO. 12, SEQ ID NO. 13, or combinations thereof.
   21. The polypeptide of any one of embodiments 1-20, wherein the polypeptide further comprises a Fc domain.
   22. The polypeptide of any one of embodiments 1-21, wherein L₁ comprises a sequence of SEQ ID NO. 9, or a combination thereof.
   23. A polypeptide comprising from the N-terminus to the C-terminus a formula of LS-X₁-L₁-X₂-L₂-X₃, LS-X₂-L₁-X₁-L₂-X₃, LS-X₁-L₁-X₃-L₂-X₂, or LS-X₂-L₁-X₃-L₂-X₁ wherein:
LS is a leader sequence;
X₁ is a PD-L1 polypeptide or a PD-L2 polypeptide;
X₂ is a B7.1 polypeptide or a B7.2 polypeptide;
X₃ is a binder, such as a Fc domain, TCR, cytokine, cytokine trap, receptor ligand, complement protein;
L₁ is absent or a polypeptide linker; and
L₂ is absent or a polypeptide linker.
   24. The polypeptide of embodiment 23, wherein:
X₁ is a PD-L1 polypeptide;
X₂ is a B7.1 polypeptide;
X₃ is a Fc domain;
L₁ is a polypeptide linker; and
L₂ is a polypeptide linker or absent.
   25. The polypeptide of embodiment 23, wherein:
X₁ is a PD-L1 polypeptide;
X₂ is a B7.2 polypeptide;
X₃ is a Fc domain;
L₁ is a polypeptide linker; and
L₂ is a polypeptide linker or absent.
   26. The polypeptide of embodiment 23, wherein:
X₁ is a PD-L2 polypeptide;
X₂ is a B7.2 polypeptide;
X₃ is a Fc domain;
L₁ is a polypeptide linker; and
L₂ is a polypeptide linker or absent.
   27. The polypeptide of embodiment 23, wherein:
X₁ is a PD-L2 polypeptide;
X₂ is a B7.1 polypeptide;
X₃ is a Fc domain;
L₁ is a polypeptide linker; and
L₂ is a polypeptide linker or absent.
   28. The polypeptide of any one of embodiments 23-27, wherein the PD-L1 polypeptide comprises a PD-L1 extracellular domain.
   29. The polypeptide of embodiment 28, wherein the PD-L1 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 2 or comprises the sequence of SEQ ID NO 2.
   30. The polypeptide of any one of embodiments 23-27, wherein the PD-L2 polypeptide comprises a PD-L2 extracellular domain.
   31. The polypeptide of embodiment 30, wherein the PD-L2 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 4 or comprises the sequence of SEQ ID NO 4.
   32. The polypeptide of any one of embodiments 23-31, wherein the B7.1 polypeptide comprises a B7.1 extracellular domain.
   33. The polypeptide of embodiment 32, wherein the B7.1 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 6 or comprises the sequence of SEQ ID NO 6.
   34. The polypeptide of any one of embodiments 23-31, wherein the B7.2 polypeptide comprises a B7.2 extracellular domain.
   35. The polypeptide of embodiment 34, wherein the B7.2 polypeptide comprises an amino acid sequence having at least 85%, 86%, 97%, 88%, 89%, 90%, 91%, 92%, 93%, 94%, 95%, 96%, 97%, 98%, or 990% identity to SEQ ID NO 8 or comprises the sequence of SEQ ID NO 8.
   36. The polypeptide of any one of embodiments 23-35, wherein the Fc domain is an Ig Fc domain selected from the group consisting essentially of human IgG, IgG₁, IgG₂, IgG₃, IgG₄, IgM, IgA, IgA₁, IgA₂, IgE, or IgD.
   37. The polypeptide of embodiment 36, wherein the Fc domain comprises an amino acid sequence having at least 85%, 86%, 97%, 88%, 89%, 90%, 91%, 92%, 93%, 94%, 95%, 96%, 97%, 98%, or 99% identity to SEQ ID NO 14 or comprises the sequence of SEQ ID NO 14.
   38. The polypeptide of embodiment 23, wherein the polypeptide comprises an amino acid sequence having at least 75%, 76%, 77%, 78%, 79%, 80%, 81%, 82%, 83%, 84%, 85%, 86%, 97%, 88%, 89%, 90%, 91%, 92%, 93%, 94%, 95%, 96%, 97%, 98%, or 99% identity to SEQ ID NO 17 or comprises the sequence of SEQ ID NO 17.
   39. The polypeptide of embodiment 23, wherein the polypeptide comprises an amino acid sequence having at least 75%, 76%, 77%, 78%, 79%, 80%, 81%, 82%, 83%, 84%, 85%, 86%, 97%, 88%, 89%, 90%, 91%, 92%, 93%, 94%, 95%, 96%, 97%, 98%, or 99% identity to SEQ ID NO 18 or comprises the sequence of SEQ ID NO 18.
   40. A nucleic acid molecule encoding a polypeptide of any one of embodiments 1-39.
   41. The nucleic acid molecule of embodiment 40, wherein the nucleic acid molecule comprises of a nucleic acid sequence having at least 60%, 65%, 70⁰/c, 75%, 80%, 85%, 95%, 96%, 97%, 98%, 99%, or 100% identity to SEQ ID NO 21 or SEQ ID NO 22.
   42. A vector comprising the nucleic acid molecule of embodiments 40 or 41.
   43. A plasmid comprising the nucleic acid molecule of embodiments 40 or 41 or a composition comprising an encapsulated nucleic acid molecule of embodiments 40 or 41.
   44. A pharmaceutical composition comprising the polypeptide of any one of embodiments 1-39.
   45. A pharmaceutical composition comprising a nucleic acid molecule encoding the polypeptide of any one of embodiments 1-39.
   46. A recombinant virus comprising a nucleic acid molecule encoding the polypeptide of any one of embodiments 1-39.
   47. The recombinant virus of embodiment 46, wherein said virus is a recombinant adenovirus.
   48. The recombinant virus of embodiment 47, wherein said recombinant adenovirus is replication-incompetent.
   49. A cell comprising or expressing the polypeptide of any one of embodiments 1-39.
   50. A cell comprising a nucleic acid molecule encoding the polypeptide of any one of embodiments 1-39.
   51. The cell of embodiments 49 or 50, wherein the cell further comprises a chimeric antigen receptor (“CAR”).
   52. The cell of any one of embodiments 49-51, wherein the cell is an immune cell.
   53. The cell of embodiment 52, wherein the immune cell is a T-cell, a monocyte, a NK cell, a B-cell or a myeloid cell.
   54. The cell of any one of embodiments 49-51, wherein the cell is a T-cell.
   55. A method for producing the cell of embodiments 49-54, the method comprising contacting the cell with a vector comprising a nucleic acid molecule encoding for the polypeptide of any one of embodiments 1-39.
   56. The method of embodiment 55, wherein the vector is a plasmid or virus.
   57. The method of embodiment 56, wherein the virus is an adenovirus or lentivirus.
   58. The method of embodiment 56, wherein the adenovirus is a replication-incompetent or replication-competent adenovirus.
   59. The method of any one of embodiments 55-58, wherein the contacting comprises transducing or transfecting the cell with a vector.
   60. The method of any one of embodiments 55-58, wherein the vector further comprises a nucleic acid molecule encoding for a chimeric antigen receptor or a tumor antigen.
   61. A method of producing a polypeptide of any one of embodiments 1-39, the method comprising transducing or transfecting a cell with a vector comprising a nucleic acid molecule encoding for the polypeptide under conditions sufficient to make the polypeptide.
   62. The method of embodiment 61, wherein the cell is transduced or transfected in vivo in a subject.
   63. The method of embodiment 61, wherein the cell is transduced or transfected ex vivo.
   64. The method of any one of embodiments 60-62, wherein the cell is an immune cell.
   65. The method of embodiment 64, wherein the immune cell is a T-cell, a monocyte, a NK cell, a B-cell or a myeloid cell
   66. The method of embodiment 64, wherein the immune cell is a T-cell.
   67. The method of any one of embodiments 61-66, wherein the vector further comprises a nucleic acid molecule encoding for a chimeric antigen receptor, any antigen, or any immunomodulatory protein.
   68. The method of embodiment 67, wherein the antigen is selected from the group consisting of tumor antigen, viral antigen, bacterial antigen, or microbial antigen.
   69. The method of any one of embodiments 61-68, wherein the vector is a plasmid or a virus.
   70. The method of embodiment 69, wherein the virus is an adenovirus or lentivirus, such as a pseudotyped lentivirus.
   71. The method of embodiment 70, wherein the adenovirus is a replication-incompetent or replication-competent adenovirus.
   72. A method for modulating an immune response in a patient, the method comprising administering to the patient a pharmaceutical composition comprising the polypeptide of any one of embodiments 1-39 or a vector comprising a nucleic acid molecule encoding for the polypeptide of any one of embodiments 1-39.
   73. The method of embodiment 72, wherein the vector is a plasmid or a virus.
   74. The method of embodiment 73, wherein the virus is an adenovirus or lentivirus, such as a pseudotyped lentivirus.
   75. The method of embodiment 74, wherein the adenovirus is a replication-incompetent or replication-competent adenovirus.
   76. The method of any one of embodiments 72-75, wherein the polypeptide or the expression of the polypeptide enhances T-cell activation.
   77. The method of any one of embodiments 72-76, wherein the vector further comprises a nucleic acid molecule encoding for a chimeric antigen receptor and/or a tumor antigen.
   78. A method of treating a cancer in a subject, the method comprising administering to the subject a pharmaceutical composition comprising the polypeptide of any one of embodiments 1-39 or a vector comprising a nucleic acid molecule encoding for the polypeptide of any one of embodiments 1-39.
   79. The method of embodiment 78, wherein the cancer is as provided for herein.
   80. The method of any one of embodiments 78-79 wherein the vector is a plasmid or a virus.
   81. The method of embodiment 80, wherein the virus is an adenovirus or lentivirus, such as pseudotyped lentivirus.
   82. The method of embodiment 81, wherein the adenovirus is a replication-incompetent or replication-competent adenovirus.
   83. The method of any one of embodiments 78-82, wherein the method further comprises the use of at least one additional therapeutic, such as an anti-cancer therapeutic, including but not limited to chemotherapy, radiation therapy, targeted therapy, immunotherapy, stem cell or bone marrow transplant therapy, hormone therapy, photodynamic therapy, or any combination thereof.
   84. A method of treating a disease or disorder, such as a viral infection, bacterial infection or fungal infection, such as those provided for herein, the method comprising administering to the subject a pharmaceutical composition comprising the polypeptide of any one of embodiments 1-39 or a vector comprising a nucleic acid molecule encoding for the polypeptide of any one of embodiments 1-39.

### EXAMPLES

The following examples are illustrative, but not limiting, of the compounds, compositions, and methods described herein. Other suitable modifications and adaptations known to those skilled in the art are within the scope of the following examples.

### Example 1: Dual Checkpoint Inhibitor Binds Both Intended Targets

Recombinant polypeptides as provided for herein will be produced via methods as provided for herein. To ensure the polypeptide binds to both intended targets (e.g., PD1 and CTLA-4), engineered mammalian cell lines will be used. Groups will include, for example, PD1 positive CTLA-4 negative cells, PD1 negative CTLA-4 positive cells, and PD1 negative CTLA-4 negative cells. Binding will be confirmed via traditional methods. For example, a radiolabeled polypeptide can be generated, and binding determined via a radiolabeled binding assay. Alternatively, in some embodiments the polypeptide provided comprises a human IgG domain. Thus, the different cell lines can be treated with recombinant polypeptide, the cells fixed, and then binding determined via microscopy using fluorophore labeled secondary antibodies to human IgG. Similarly, after treatment, cell lysates for the different groups can be generated and the binding confirmed via western blot analysis using secondary antibodies to human IgG.

In some embodiments, nucleic acid molecules encoding for the polypeptide as provided for herein are utilized. To ensure proper translation of the polypeptides followed by binding to both intended targets, mammalian cell lines will be used as described above. The nucleic acid molecules will be expressed in the different groups via recognized methods, for example transfection using a lipid-based technology, liposomes, or electroporation. Cell groups will be allowed to incubate for an appropriate period of time to allow for expression of the polypeptides. Expression will be determined via traditional methods, for example cell groups may be lysed and expression of the polypeptide determined via western blot analysis using an antibody targeting the PD-L domain, the B7 domain, or the IgG domain if present. Interaction of the expressed polypeptide with the intended targets will be determined via traditional methods. For example, the targets may be immunoprecipitated from cell lysates and an interaction determined via Co-IP western blot analysis. Alternatively, antibodies targeting the polypeptide and the target may be utilized and an interaction determined via microscopy using an assay such as a proximity ligation assay or the like.

### Example 2: Use of a Pharmacological Composition Comprising the Polypeptide to Treat Cancer

Pharmacological compositions comprising a polypeptide as provided for herein will be prepared and will be administered to a subject suffering from cancer. Administration of the polypeptide will diminish the cancer cell's ability to evade the subject's immune system, thereby enhancing the activity of the immune system against the cancer cell, thereby treating the cancer.

### Example 3: Use of a Pharmacological Composition Comprising a Virus to Treat Cancer

Pharmacological compositions comprising a virus as provided for herein will be prepared and will be administered to a subject suffering from cancer. The virus comprises a nucleic acid molecule encoding for a polypeptide as provided for herein. The virus will transduce the target cell, which will result in expression of the polypeptide. Expression of the polypeptide will diminish the cancer cell's ability to evade the subject's immune system, thereby enhancing the activity of the immune system against the cancer cell, thereby treating the cancer. The vector can also comprise a nucleic acid molecule encoding for a tumor antigen. The vector can also comprise a nucleic acid molecule encoding for a CAR.

### Example 4: Dual Checkpoint Inhibitor Represses CTLA-4 and PD-1 Immunosuppressive Signals

A dual checkpoint inhibitor composition comprising, from the N-terminus to the C-terminus, a PD-L2 domain linked to a B7.1 domain plus a Fc domain, is tested via ELISA to determine the relative binding of CLTA-4 and PD-1.

First, the composition's binding of CTLA-4 and PD-1 was tested against four different additional compositions: a B7.1 domain only plus Fc construct, a PD-L2 domain only plus Fc construct, a commercially available CTLA-4 binder, and a commercially available PD-1 binder. Results are listed in Table 2 below and show that the dual checkpoint inhibitor composition successfully binds both CTLA-4 and PD-1, while the other constructs bind only CTLA-4 or PD-1.

TABLE 2

| | CLTA-4 Relative | PD-1 Relative |
|---|---|---|
| Composition | Binding | Binding |
| PD-L2/B7.1 Dual Checkpoint | 1.651 | 3.367 |
| B7.1 only | 3.259 | 0.004 |
| PD-L2 only | 0.008 | 1.768 |
| Commercial CTLA-4 binder | 2.647 | 0.007 |
| Commercial PD-1 binder | 0.025 | 1.825 |

Next, the composition's binding of CTLA4 and PD-1 was tested against a different composition comprising the domains in a reverse order: a B7.1 domain linked to a PD-L2 domain plus an Fc domain. Additional control compounds used were the B7.1 only and commercial binders used above. Results are listed in Table 3 and show that while the PD-L2/B7.1 Dual Checkpoint composition continues to show both CTLA-4 and PD-1 binding, the reverse B7.1/PD-L2 composition only binds CLTA-4.

TABLE 3

| | CLTA-4 Relative | PD-1 Relative |
|---|---|---|
| Composition | Binding | Binding |
| PD-L2/B7.1 Dual Checkpoint | 0.266 | 0.088 |
| B7.1/PD-L2 composition | 0.548 | 0 |
| B7.1 only | 0.236 | 0 |
| Commercial CTLA-4 binder | 1.075 | 0 |
| Commercial PD-1 binder | 0 | 0.400 |

### Example 5: In Vivo Antigen Specific T Cell Response Stimulated by Dual Checkpoint Inhibitor

To determine the effect of dual check point inhibitor in vivo, the composition comprising a PD-L2 domain linked to a B7.1 domain plus a Fc domain used in Example 4 was injected into mice along with a model antigen (ovalbumin). A vehicle control group, a vehicle control plus ovalbumin group, and a PD-L2/B7.1 Dual Checkpoint plus ovalbumin group were administered intravenously to different group of C57BL/6 mice at day 0, following by a second administration at day 21. The animals were acclimated a minimum of 3 days prior to the start of the study and were housed in microisolators in a 12:12 light/dark cycle. Animals were maintained with a standard rodent chow diet with water and food provided ad libitum. The spleens were harvested at day 28 and ELIspot assays were run to determine the levels of antigen reactive T cells. The results are listed in Table 4 and show that while the PD-L2/B7.1 Dual Checkpoint plus ovalbumin group had a much higher level of reactive T cells compared to the control groups.

TABLE 4

| Group | IFN-g SFC/10e⁶ |
|---|---|
| Vehicle control | 0 |
| Control + antigen | 40 |
| PD-L2/B7.1 Dual | 97 |
| Checkpoint + antigen | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A polypeptide comprising from the N-terminus to the C-terminus a formula of X₁-L₁-X₂ or a formula of X₂-L₁-X₁ wherein:
X₁ is an immune checkpoint polypeptide, or an active fragment thereof, such as, but not limited, to, a CTLA-4 polypeptide, a PD-1 polypeptide, a PD-L1 polypeptide, a PD-L2 polypeptide, a TIM3 polypeptide, a LAG3 polypeptide, a VISTA polypeptide, a SINGLEC7 polypeptide, a SINGLEC9 polypeptide, a TIGIT polypeptide, a CD96 polypeptide, a BTLA polypeptide, a B7H3 polypeptide, a B7H4 polypeptide, a CD155 polypeptide, a HHLA2 polypeptide, a BTN3A1 polypeptide, or an active fragment thereof;
X₂ is a B7.1 polypeptide, or a B7.2 polypeptide; and
L₁ is a polypeptide linker.

2. The polypeptide of claim 1, wherein the polypeptide comprising from the N-terminus to the C-terminus a formula of X₁-L₁-X₂.

3. The polypeptide of claim 1, wherein the polypeptide comprising from the N-terminus to the C-terminus a formula of X₂-L₁-X₁.

4. The polypeptide of any one of claims 1-3, wherein:
X₁ is a PD-L1 polypeptide; and
X₂ is a B7.1 polypeptide.

5. The polypeptide of any one of claims 1-3, wherein:
X₁ is a PD-L1 polypeptide; and
X₂ is a B7.2 polypeptide.

6. The polypeptide of any one of claims 1-3, wherein:
X₁ is a PD-L2 polypeptide; and
X₂ is a B7.1 polypeptide.

7. The polypeptide of any one of claims 1-3, wherein:
X₁ is a PD-L2 polypeptide; and
X₂ is a B7.2 polypeptide.

8. The polypeptide of any one of claims 1-7, wherein the PD-L1 polypeptide comprises a PD-L1 extracellular domain.

9. The polypeptide of claim 10, wherein the PD-L1 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 2 or comprises the sequence of SEQ ID NO 2.

10. The polypeptide of any one of claims 1-7, wherein the PD-L2 polypeptide comprises a PD-L2 extracellular domain.

11. The polypeptide of claim 10, wherein the PD-L2 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 4.

12. The polypeptide of claim 11, wherein the PD-L2 polypeptide comprises of SEQ ID NO 4.

13. The polypeptide of any one of claims 1-12, wherein the B7.1 polypeptide comprises a B7.1 extracellular domain.

14. The polypeptide of claim 13, wherein the B7.1 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 6 or comprises the sequence of SEQ ID NO 6.

15. The polypeptide of any one of claims 1-12, wherein the B7.2 polypeptide comprises a B7.2 extracellular domain.

16. The polypeptide of claim 15, wherein the B7.2 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 8 or comprises the sequence of SEQ ID NO 8.

17. The polypeptide of any one of claims 1-16, wherein the polypeptide comprises a leader sequence.

18. The polypeptide of claim 17, wherein the leader sequence is at the N-terminus of the polypeptide.

19. The polypeptide of claim 17 or 18, wherein the leader sequence is a cell membrane targeting polypeptide.

20. The polypeptide of any one of claims 17-19, wherein the leader sequence is a sequence of SEQ ID NO. 10, SEQ ID NO. 11, SEQ ID NO. 12, SEQ ID NO. 13, or combinations thereof.

21. The polypeptide of any one of claims 1-20, wherein the polypeptide further comprises a Fc domain.

22. The polypeptide of any one of claims 1-21, wherein L₁ comprises a sequence of SEQ ID NO. 9, or a combination thereof.

23. A polypeptide comprising from the N-terminus to the C-terminus a formula of LS-X₁-L₁−X₂-L₂-X₃, LS-X₂-L₁-X₁-L₂-X₃, LS-Xt-L₁-X₃-L₂-X₂, or LS-X₂-L₁-X₃-L₂-X₁ wherein:
LS is a leader sequence;
X₁ is a PD-L1 polypeptide or a PD-L2 polypeptide;
X₂ is a B7.1 polypeptide or a B7.2 polypeptide;
X₃ is a binder, such as a Fc domain, TCR, cytokine, cytokine trap, receptor ligand, complement protein;
L₁ is absent or a polypeptide linker; and
L₂ is absent or a polypeptide linker.

24. The polypeptide of claim 23, wherein:
X₁ is a PD-L1 polypeptide;
X₂ is a B7.1 polypeptide;
X₃ is a Fc domain;
L₁ is a polypeptide linker; and
L₂ is a polypeptide linker or absent.

25. The polypeptide of claim 23, wherein:
X₁ is a PD-L1 polypeptide;
X₂ is a B7.2 polypeptide;
X₃ is a Fc domain;
L₁ is a polypeptide linker; and
L₂ is a polypeptide linker or absent.

26. The polypeptide of claim 23, wherein:
X₁ is a PD-L2 polypeptide;
X₂ is a B7.2 polypeptide;
X₃ is a Fc domain;
L₁ is a polypeptide linker; and
L₂ is a polypeptide linker or absent.

27. The polypeptide of claim 23, wherein:
Xⱼ is a PD-L2 polypeptide;
X₂ is a B7.1 polypeptide;
X₃ is a Fc domain;
L₁ is a polypeptide linker; and
L₂ is a polypeptide linker or absent.

28. The polypeptide of any one of claims 23-27, wherein the PD-L1 polypeptide comprises a PD-L1 extracellular domain.

29. The polypeptide of claim 28, wherein the PD-L1 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 2 or comprises the sequence of SEQ ID NO 2.

30. The polypeptide of any one of claims 23-27, wherein the PD-L2 polypeptide comprises a PD-L2 extracellular domain.

31. The polypeptide of claim 30, wherein the PD-L2 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 4 or comprises the sequence of SEQ ID NO 4.

32. The polypeptide of any one of claims 23-31, wherein the B7.1 polypeptide comprises a B7.1 extracellular domain.

33. The polypeptide of claim 32, wherein the B7.1 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 6 or comprises the sequence of SEQ ID NO 6.

34. The polypeptide of any one of claims 23-31, wherein the B7.2 polypeptide comprises a B7.2 extracellular domain.

35. The polypeptide of claim 34, wherein the B7.2 polypeptide comprises an amino acid sequence having at least 85%, 86%, 97%, 88%, 89%, 90%, 91%, 92%, 93%, 94%, 95%, 96%, 97%, 98%, or 99% identity to SEQ ID NO 8 or comprises the sequence of SEQ ID NO 8.

36. The polypeptide of any one of claims 23-35, wherein the Fc domain is an Ig Fc domain selected from the group consisting essentially of human IgG, IgG₁, IgG₂, IgG₃, IgG₄, IgM, IgA, IgA₁, IgA₂, IgE, or IgD.

37. The polypeptide of claim 36, wherein the Fc domain comprises an amino acid sequence having at least 85%, 86%, 97%, 88%, 89%, 90%, 91%, 92%, 93%, 94%, 95%, 96%, 97%, 98%, or 99% identity to SEQ ID NO 14 or comprises the sequence of SEQ ID NO 14.

38. The polypeptide of claim 23, wherein the polypeptide comprises an amino acid sequence having at least 75%, 76%, 77%, 78%, 79%, 80%, 81%, 82%, 83%, 84%, 85%, 86%, 97%, 88%, 89%, 90%, 91%, 92%, 93%, 94%, 95%, 96%, 97%, 98%, or 99% identity to SEQ ID NO 17 or comprises the sequence of SEQ ID NO 17.

39. The polypeptide of claim 23, wherein the polypeptide comprises an amino acid sequence having at least 75%, 76%, 77%, 78%, 79%, 80%, 81%, 82%, 83%, 84%, 85%, 86%, 97%, 88%, 89%, 90%, 91%, 92%, 93%, 94%, 95%, 96%, 97%, 98%, or 99% identity to SEQ ID NO 18 or comprises the sequence of SEQ ID NO 18.

40. A nucleic acid molecule encoding a polypeptide of any one of claims 1-39.

41. The nucleic acid molecule of claim 40, wherein the nucleic acid molecule comprises of a nucleic acid sequence having at least 60%, 65%, 70%, 75%, 80%, 85%, 95%, 96%, 97%, 98%, 99%, or 100% identity to SEQ ID NO 21 or SEQ ID NO 22.

42. A vector comprising the nucleic acid molecule of claims 40 or 41.

43. A plasmid comprising the nucleic acid molecule of claims 40 or 41 or a composition comprising an encapsulated nucleic acid molecule of claims 40 or 41.

44. A pharmaceutical composition comprising the polypeptide of any one of claims 1-39.

45. A pharmaceutical composition comprising a nucleic acid molecule encoding the polypeptide of any one of claims 1-39.

46. A recombinant virus comprising a nucleic acid molecule encoding the polypeptide of any one of claims 1-39.

47. The recombinant virus of claim 46, wherein said virus is a recombinant adenovirus.

48. The recombinant virus of claim 47, wherein said recombinant adenovirus is replication-incompetent.

49. A cell comprising or expressing the polypeptide of any one of claims 1-39.

50. A cell comprising a nucleic acid molecule encoding the polypeptide of any one of claims 1-39.

51. The cell of claims 49 or 50, wherein the cell further comprises a chimeric antigen receptor (“CAR”).

52. The cell of any one of claims 49-51, wherein the cell is an immune cell.

53. The cell of claim 52, wherein the immune cell is a T-cell, a monocyte, a NK cell, a B-cell or a myeloid cell.

54. The cell of any one of claims 49-51, wherein the cell is a T-cell.

55. A method for producing the cell of claims 49-54, the method comprising contacting the cell with a vector comprising a nucleic acid molecule encoding for the polypeptide of any one of claims 1-39.

56. The method of claim 55, wherein the vector is a plasmid or virus.

57. The method of claim 56, wherein the virus is an adenovirus or lentivirus.

58. The method of claim 56, wherein the adenovirus is a replication-incompetent or replication-competent adenovirus.

59. The method of any one of claims 55-58, wherein the contacting comprises transducing or transfecting the cell with a vector.

60. The method of any one of claims 55-58, wherein the vector further comprises a nucleic acid molecule encoding for a chimeric antigen receptor or a tumor antigen.

61. A method of producing a polypeptide of any one of claims 1-39, the method comprising transducing or transfecting a cell with a vector comprising a nucleic acid molecule encoding for the polypeptide under conditions sufficient to make the polypeptide.

62. The method of claim 61, wherein the cell is transduced or transfected in viv) in a subject.

63. The method of claim 61, wherein the cell is transduced or transfected ex vivo.

64. The method of any one of claims 60-62, wherein the cell is an immune cell.

65. The method of claim 64, wherein the immune cell is a T-cell, a monocyte, a NK cell, a B-cell or a myeloid cell

66. The method of claim 64, wherein the immune cell is a T-cell.

67. The method of any one of claims 61-66, wherein the vector further comprises a nucleic acid molecule encoding for a chimeric antigen receptor, any antigen, or any immunomodulatory protein.

68. The method of claim 67, wherein the antigen is selected from the group consisting of tumor antigen, viral antigen, bacterial antigen, or microbial antigen.

69. The method of any one of claims 61-68, wherein the vector is a plasmid or a virus.

70. The method of claim 69, wherein the virus is an adenovirus or lentivirus, such as a pseudotyped lentivirus.

71. The method of claim 70, wherein the adenovirus is a replication-incompetent or replication-competent adenovirus.

72. A method for modulating an immune response in a patient, the method comprising administering to the patient a pharmaceutical composition comprising the polypeptide of any one of claims 1-39 or a vector comprising a nucleic acid molecule encoding for the polypeptide of any one of claims 1-39.

73. The method of claim 72, wherein the vector is a plasmid or a virus.

74. The method of claim 73, wherein the virus is an adenovirus or lentivirus, such as a pseudotyped lentivirus.

75. The method of claim 74, wherein the adenovirus is a replication-incompetent or replication-competent adenovirus.

76. The method of any one of claims 72-75, wherein the polypeptide or the expression of the polypeptide enhances T-cell activation.

77. The method of any one of claims 72-76, wherein the vector further comprises a nucleic acid molecule encoding for a chimeric antigen receptor and/or a tumor antigen.

78. A method of treating a cancer in a subject, the method comprising administering to the subject a pharmaceutical composition comprising the polypeptide of any one of claims 1-39 or a vector comprising a nucleic acid molecule encoding for the polypeptide of any one of claims 1-39.

79. The method of claim 78, wherein the cancer is as provided for herein.

80. The method of any one of claims 78-79 wherein the vector is a plasmid or a virus.

81. The method of claim 80, wherein the virus is an adenovirus or lentivirus, such as pseudotyped lentivirus.

82. The method of claim 81, wherein the adenovirus is a replication-incompetent or replication-competent adenovirus.

83. The method of any one of claims 78-82, wherein the method further comprises the use of at least one additional therapeutic, such as an anti-cancer therapeutic, including but not limited to chemotherapy, radiation therapy, targeted therapy, immunotherapy, stem cell or bone marrow transplant therapy, hormone therapy, photodynamic therapy, or any combination thereof.

84. A method of treating a disease or disorder, such as a viral infection, bacterial infection or fungal infection, such as those provided for herein, the method comprising administering to the subject a pharmaceutical composition comprising the polypeptide of any one of claims 1-39 or a vector comprising a nucleic acid molecule encoding for the polypeptide of any one of claims 1-39.


SEQUENCE LISTING (44 sequences)

SEQ ID NO: 1 - protein, 290 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 238 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 3 - protein, 273 aa, synthetic construct
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 4 - protein, 220 aa, synthetic construct
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 5 - protein, 288 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 6 - protein, 214 aa, synthetic construct
LSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPD

SEQ ID NO: 7 - protein, 329 aa, synthetic construct
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 8 - protein, 224 aa, synthetic construct
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 9 - protein, 19 aa, synthetic construct
GSGSGGGSGSGGGSGSGSG

SEQ ID NO: 10 - protein, 19 aa, synthetic construct
MIFLLLMLSLELQLHQIAA

SEQ ID NO: 11 - protein, 18 aa, synthetic construct
MRIFAVFIFMTYWHLLNA

SEQ ID NO: 12 - protein, 27 aa, synthetic construct
MGHTRRQGTSPSKCPYLNFFQLLVLAG

SEQ ID NO: 13 - protein, 23 aa, synthetic construct
MDPQCTMGLSNILFVMAFLLSGA

SEQ ID NO: 14 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 453 aa, synthetic construct
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTGSGSGGGSGSGGGSGSGSGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPD

SEQ ID NO: 16 - protein, 480 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERGSGSGGGSGSGGGSGSGSAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 17 - protein, 680 aa, synthetic construct
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTGSGSGGGSGSGGGSGSGSGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 707 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERGSGSGGGSGSGGGSGSGSAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1359 bp, synthetic construct
  source 1..1359: mol_type = other DNA
atgatttttctcttgcttatgctttctcttgagttgcaacttcatcagattgccgccctttttaccgtcacagtccctaaagaactttacataatcgagcacggtagcaacgtaacgcttgaatgtaactttgacacggggagtcacgtcaatcttggagctattacggcgtctcttcagaaggtggaaaacgatacgagccctcaccgggagcgcgctaccctgcttgaagaacagttgccgcttggaaaagcatcctttcacataccgcaggttcaagttcgagatgaaggccaataccaatgcataattatttatggagtcgcctgggactataagtacctgacattgaaggttaaagcgagctataggaagatcaatactcacatactgaaggtcccggagactgatgaagtggaattgacttgtcaggcaacagggtatcctttggcggaggttagttggcctaatgtttccgtgcccgcgaacacatctcattcaaggacaccagagggcctgtatcaggtgacctccgttctgcgcctgaagccaccgccaggtaggaactttagctgcgtcttctggaatactcatgttcgagaactcacacttgcttcaatcgaccttcagtcccagatggagcctagaactcatcctaccggatcaggtagtggcggcggaagcgggagtggcggaggctccgggtctggttccggcctgtcacacttttgttctggcgtaatccacgtcactaaggaggttaaagaggtagccaccctttcttgtggtcataatgtctctgtggaagaacttgctcaaacaagaatctactggcaaaaggagaagaaaatggtcttgacgatgatgagcggtgacatgaacatatggccagagtacaagaatcgcaccattttcgacataaccaacaatctttcaatcgtaatactggccctcaggccgtctgatgaaggtacgtatgagtgcgttgtcttgaaatacgaaaaagacgccttcaagcgagaacatctggcggaggtgacactttctgttaaagccgatttcccaactccttctatttcagattttgaaatcccgacctctaacatccggagaattatctgcagcacgtcaggggggtttcctgaaccacatttgtcttggcttgagaatggcgaagaattgaacgccatcaacaccaccgttagtcaagacccagagacggaactgtatgccgtttcaagtaagctcgattttaatatgacaactaaccactccttcatgtgtctgataaaatatggacatctgcgggtaaatcagacatttaactggaacacgacaaaacaagagcatttcccagat

SEQ ID NO: 20 - DNA, 1440 bp, synthetic construct
  source 1..1440: mol_type = other DNA
atgcgcatattcgcagtctttatatttatgacttattggcatcttcttaatgcatttactgtcactgttcccaaggatctgtacgttgtagagtacggctccaacatgactatagagtgcaaatttccagtcgagaaacagctcgacttggcagcactcattgtgtactgggaaatggaagacaaaaacatcattcagttcgtccacggggaggaggacttgaaagttcaacactcatcctatcgccaacgagcccgcctgttgaaggaccagctttctctcggtaacgctgcacttcagattacggatgttaagttgcaggatgcaggagtctaccgatgtatgattagttacggcggtgccgattacaagaggatcactgttaaagtcaacgctccttacaataaaataaaccaacggattctcgtggtagatcctgtgacttccgagcacgaattgacgtgccaagcagaaggatacccgaaagccgaggtgatctggacaagctccgatcatcaagtcctctcagggaagacgacgaccacgaactctaagagggaagagaagctgttcaacgtaacatcaacgctccgcattaacacgactactaatgagatattctactgcactttccggcgactcgatccggaggaaaaccatacagcggagcttgtgattccggaacttccactcgcgcatcctccgaacgagcggggatcaggtagtggcggcggatccgggagtggcgggggcagcgggtctggttccgcaccgttgaaaatacaagcgtacttcaacgaaactgcagaccttccgtgtcaattcgcgaactcacaaaatcaatcccttagtgagcttgtagtattttggcaggaccaggaaaatctcgtcctgaacgaagtttatctcggtaaagagaagttcgacagcgtacatagtaaatacatgggcagaacttctttcgatagtgatagttggacgctcaggttgcataacctccagatcaaggataaaggcctgtatcaatgtattatccaccacaaaaaacccacaggcatgatacggatacatcaaatgaactctgaactgtcagtcttggcaaatttcagtcaacctgagattgtacctatcagcaacattaccgagaatgtctacattaatctcacttgctccagcatacatgggtatccagaacctaagaaaatgtccgtactccttagaacgaagaactcaactattgagtacgacggagtaatgcaaaaatcccaagataatgtcacagagctttatgatgtgagtatatcactcagtgtatcattccctgatgtgacttcaaatatgactattttctgtatattggagacagataaaacaaggctgttgagtagtcctttctcaatagagttggaggacccacaaccgccacccgaccacatcccc

SEQ ID NO: 21 - DNA, 2040 bp, synthetic construct
  source 1..2040: mol_type = other DNA
atgatttttctcttgcttatgctttctcttgagttgcaacttcatcagattgccgccctttttaccgtcacagtccctaaagaactttacataatcgagcacggtagcaacgtaacgcttgaatgtaactttgacacggggagtcacgtcaatcttggagctattacggcgtctcttcagaaggtggaaaacgatacgagccctcaccgggagcgcgctaccctgcttgaagaacagttgccgcttggaaaagcatcctttcacataccgcaggttcaagttcgagatgaaggccaataccaatgcataattatttatggagtcgcctgggactataagtacctgacattgaaggttaaagcgagctataggaagatcaatactcacatactgaaggtcccggagactgatgaagtggaattgacttgtcaggcaacagggtatcctttggcggaggttagttggcctaatgtttccgtgcccgcgaacacatctcattcaaggacaccagagggcctgtatcaggtgacctccgttctgcgcctgaagccaccgccaggtaggaactttagctgcgtcttctggaatactcatgttcgagaactcacacttgcttcaatcgaccttcagtcccagatggagcctagaactcatcctaccggatcaggtagtggcggcggaagcgggagtggcggaggctccgggtctggttccggcctgtcacacttttgttctggcgtaatccacgtcactaaggaggttaaagaggtagccaccctttcttgtggtcataatgtctctgtggaagaacttgctcaaacaagaatctactggcaaaaggagaagaaaatggtcttgacgatgatgagcggtgacatgaacatatggccagagtacaagaatcgcaccattttcgacataaccaacaatctttcaatcgtaatactggccctcaggccgtctgatgaaggtacgtatgagtgcgttgtcttgaaatacgaaaaagacgccttcaagcgagaacatctggcggaggtgacactttctgttaaagccgatttcccaactccttctatttcagattttgaaatcccgacctctaacatccggagaattatctgcagcacgtcaggggggtttcctgaaccacatttgtcttggcttgagaatggcgaagaattgaacgccatcaacaccaccgttagtcaagacccagagacggaactgtatgccgtttcaagtaagctcgattttaatatgacaactaaccactccttcatgtgtctgataaaatatggacatctgcgggtaaatcagacatttaactggaacacgacaaaacaagagcatttcccagatgataaaacccatacctgtcctccttgccctgcccccgaactgctgggtgggcctagcgtgttccttttcccgcccaagccgaaggacacactcatgatcagtcgaacgcccgaagttacgtgcgtggtcgtagatgtgtcccacgaagatccagaggtgaagtttaattggtacgttgatggtgtagaggtacataatgccaaaacaaagccacgcgaagagcagtataatagcacctatagagtagttagcgttctgacagttctccaccaggattggcttaacggcaaagagtacaagtgtaaagtatcaaacaaagcacttcctgcacccattgaaaaaactatatctaaggccaaaggccaacctagggaaccgcaggtctacacgctgcccccgagcagagacgagcttacgaagaaccaagtctcactgacgtgtcttgtcaagggtttttaccccagtgacattgcagtagagtgggaatcaaatgggcaaccagaaaacaactataaaactacaccaccagtcttggacagtgatggatcattcttcttgtattcaaaacttacggttgacaagagccgctggcaacagggcaacgtgttcagctgctccgtgatgcatgaggcgcttcacaaccattacacccaaaagagtctttcactctctcccggaaaa

SEQ ID NO: 22 - DNA, 2121 bp, synthetic construct
  source 1..2121: mol_type = other DNA
atgcgcatattcgcagtctttatatttatgacttattggcatcttcttaatgcatttactgtcactgttcccaaggatctgtacgttgtagagtacggctccaacatgactatagagtgcaaatttccagtcgagaaacagctcgacttggcagcactcattgtgtactgggaaatggaagacaaaaacatcattcagttcgtccacggggaggaggacttgaaagttcaacactcatcctatcgccaacgagcccgcctgttgaaggaccagctttctctcggtaacgctgcacttcagattacggatgttaagttgcaggatgcaggagtctaccgatgtatgattagttacggcggtgccgattacaagaggatcactgttaaagtcaacgctccttacaataaaataaaccaacggattctcgtggtagatcctgtgacttccgagcacgaattgacgtgccaagcagaaggatacccgaaagccgaggtgatctggacaagctccgatcatcaagtcctctcagggaagacgacgaccacgaactctaagagggaagagaagctgttcaacgtaacatcaacgctccgcattaacacgactactaatgagatattctactgcactttccggcgactcgatccggaggaaaaccatacagcggagcttgtgattccggaacttccactcgcgcatcctccgaacgagcggggatcaggtagtggcggcggatccgggagtggcgggggcagcgggtctggttccgcaccgttgaaaatacaagcgtacttcaacgaaactgcagaccttccgtgtcaattcgcgaactcacaaaatcaatcccttagtgagcttgtagtattttggcaggaccaggaaaatctcgtcctgaacgaagtttatctcggtaaagagaagttcgacagcgtacatagtaaatacatgggcagaacttctttcgatagtgatagttggacgctcaggttgcataacctccagatcaaggataaaggcctgtatcaatgtattatccaccacaaaaaacccacaggcatgatacggatacatcaaatgaactctgaactgtcagtcttggcaaatttcagtcaacctgagattgtacctatcagcaacattaccgagaatgtctacattaatctcacttgctccagcatacatgggtatccagaacctaagaaaatgtccgtactccttagaacgaagaactcaactattgagtacgacggagtaatgcaaaaatcccaagataatgtcacagagctttatgatgtgagtatatcactcagtgtatcattccctgatgtgacttcaaatatgactattttctgtatattggagacagataaaacaaggctgttgagtagtcctttctcaatagagttggaggacccacaaccgccacccgaccacatccccgataaaacccatacctgtcctccttgccctgcccccgaactgctgggtgggcctagcgtgttccttttcccgcccaagccgaaggacacactcatgatcagtcgaacgcccgaagttacgtgcgtggtcgtagatgtgtcccacgaagatccagaggtgaagtttaattggtacgttgatggtgtagaggtacataatgccaaaacaaagccacgcgaagagcagtataatagcacctatagagtagttagcgttctgacagttctccaccaggattggcttaacggcaaagagtacaagtgtaaagtatcaaacaaagcacttcctgcacccattgaaaaaactatatctaaggccaaaggccaacctagggaaccgcaggtctacacgctgcccccgagcagagacgagcttacgaagaaccaagtctcactgacgtgtcttgtcaagggtttttaccccagtgacattgcagtagagtgggaatcaaatgggcaaccagaaaacaactataaaactacaccaccagtcttggacagtgatggatcattcttcttgtattcaaaacttacggttgacaagagccgctggcaacagggcaacgtgttcagctgctccgtgatgcatgaggcgcttcacaaccattacacccaaaagagtctttcactctctcccggaaaa

SEQ ID NO: 23 - protein, 5 aa, synthetic construct
GGGGS

SEQ ID NO: 24 - protein, 10 aa, synthetic construct
GGGGSGGGGS

SEQ ID NO: 25 - protein, 15 aa, synthetic construct
GGGGSGGGGSGGGGS

SEQ ID NO: 26 - protein, 20 aa, synthetic construct
GGGGSGGGGSGGGGSGGGGS

SEQ ID NO: 27 - protein, 6 aa, synthetic construct
GGGGGG

SEQ ID NO: 28 - protein, 8 aa, synthetic construct
GGGGGGGG

SEQ ID NO: 29 - protein, 15 aa, synthetic construct
EAAAKEAAAKEAAAK

SEQ ID NO: 30 - protein, 4 aa, synthetic construct
EAAK

SEQ ID NO: 31 - protein, 8 aa, synthetic construct
EAAKEAAK

SEQ ID NO: 32 - protein, 12 aa, synthetic construct
EAAKEAAKEAAK

SEQ ID NO: 33 - protein, 46 aa, synthetic construct
AEAAAKEAAAKEAAAKEAAAKALEAEAAAKEAAAKEAAAKEAAAKA

SEQ ID NO: 34 - protein, 12 aa, synthetic construct
AEAAAKEAAAKA

SEQ ID NO: 35 - protein, 5 aa, synthetic construct
PAPAP

SEQ ID NO: 36 - protein, 17 aa, synthetic construct
VSQTSKLTRAETVFPDV

SEQ ID NO: 37 - protein, 6 aa, synthetic construct
PLGLWA

SEQ ID NO: 38 - protein, 6 aa, synthetic construct
RVLAEA

SEQ ID NO: 39 - protein, 10 aa, synthetic construct
EDVVCCSMSY

SEQ ID NO: 40 - protein, 8 aa, synthetic construct
GGIEGRGS

SEQ ID NO: 41 - protein, 10 aa, synthetic construct
TRHRQPRGWE

SEQ ID NO: 42 - protein, 10 aa, synthetic construct
AGNRVRRSVG

SEQ ID NO: 43 - protein, 9 aa, synthetic construct
RRRRRRRRR

SEQ ID NO: 44 - protein, 4 aa, synthetic construct
GFLG